(19) United States
(12) Patent Application Publication
(10) Pub. No.: US 2019/0071685 A1
(43) Pub. Date: Mar. 7, 2019

(54) IDENTIFICATION OF CROP MYB TRANSCRIPTION FACTORS AND THEIR USE

(71) Applicant: PURDUE RESEARCH FOUNDATION, West Lafayette, IN (US)

(72) Inventors: Stanton B. Gelvin, West Lafayette, IN (US); Nagesh N. Sardesai, West Lafayette, IN (US)

(21) Appl. No.: 16/176,936

(22) Filed: Oct. 31, 2018

Related U.S. Application Data
(63) Continuation-in-part of application No. 15/004,227, filed on Jan. 22, 2016, now Pat. No. 10,150,969.
Application No. 15/004,227 is a division of application No. 13/884,480, filed on May 9, 2013, now abandoned.
(60) Provisional application No. 61/412,684, filed on Nov. 11, 2010.
(60) Provisional application No. 61/552,127, filed on Oct. 27, 2011.

(51) Int. Cl. C12N 15/82; C07K 14/415

(52) U.S. Cl. CPC C12N 15/8205; C12N 15/8282; Y02A 40/146; C07K 14/415; C12N 15/8261

(57) ABSTRACT

Mutation of the rice ortholog of the Arabidopsis MTF1 gene results in increased rice transformation. CRISPR was used to generate several different mutations in the rice MTF1 gene. Agrobacterium-mediated transformation of these homozygous mutants indicated 5- to 10-fold increased transient and stable transformation, using GUS and luciferase assays.

### CROSS REFERENCE TO RELATED APPLICATIONS

This application is a continuation-in-part of co-pending U.S. application Ser. No. 15/004,227 filed Jan. 22, 2016, which is a Divisional of U.S. application Ser. No. 13/884,480, filed May 9, 2013, which is a U.S. nationalization under 35 U.S.C. § 371 of International Application No. PCT/US2011/059944, filed Nov. 9, 2011, which claims the benefit of priority under 35 U.S.C. § 119(e) to U.S. provisional application nos. 61/412,684, filed Nov. 11, 2010, and 61/552,127, filed Oct. 27, 2011. The disclosures of the referenced applications are incorporated herein by reference in their entireties.

### SEQUENCE LISTING

The instant application contains a Sequence Listing which has been submitted in ASCII format via EFS-Web and is hereby incorporated by reference in its entirety. Said ASCII copy, created on Oct. 31, 2018, is named 286904_SEQ_ST25.txt and is 47,999 bytes in size.

### BACKGROUND

Mutation of the rice ortholog of the Arabidopsis MTF1 gene results in increased rice transformation. CRISPR was used to generate several different mutations in the rice MTF1 gene. Agrobacterium-mediated transformation of these homozygous mutants indicated 5- to 10-fold increased transient and stable transformation, using GUS and luciferase assays.

Agrobacterium-mediated plant transformation is a highly complex process that involves genetic determinants of the bacterium and host plant cell. A problem with this process is that many plant species are highly resistant to it. This means these species of plants are not open to transgenic manipulation or genetic engineering. Current methods manipulate other genes that permit plant regeneration, but this method is not always successful for various plant species and varieties. There is a need for a new method to make these resistant plant species more open to Agrobacterium-mediated plant transformation.

A myb transcription factor designated MTF is disclosed that negatively regulates plant transformation susceptibility. An integrin domain-like protein (which is under negative regulation by MTF) is involved in Agrobacterium attachment to plant cells and, thus, is a positive mediator of transformation: plants over-expressing the integrin domain-like protein are more susceptible to transformation, whereas plants mutant for the integrin domain-like protein are less susceptible. Manipulation of these elements allows improved control of Agrobacterial transformation of plants, including in crops.

Agrobacterium-mediated plant transformation forms the basis for the modern agricultural biotechnology industry.

Agrobacterium tumefaciens causes the disease crown gall and genetically transforms numerous plant, fungal and animal species. Virulent Agrobacteria harbor a tumor-inducing (Ti) plasmid containing virulence (vir) genes required by the pathogen for transport of transferred (T-) DNA and virulence effector proteins to host cells. Induction of vir genes, processing of T-DNA from the Ti-plasmid, attachment of the bacteria to plants, and subsequent transfer of T-DNA and Vir proteins to host cells are complex processes. Numerous studies have elucidated the events governing these processes in the bacterium, but relatively little is known about the plant contribution to transformation.

Although Agrobacterium has a broad host range, many economically important plants remain recalcitrant to transformation. Scientists have used a variety of techniques to identify plant genes that are involved in Agrobacterium-mediated transformation. Among these, forward and reverse genetic screens revealed more than 125 Arabidopsis and tobacco genes involved in transformation. Collectively these lines, designated “rat” (resistant to Agrobacterium transformation), reflected their attenuated response to transformation. The genes identified represent steps necessary for successful transformation, including bacterial attachment/biofilm formation, T-DNA and Vir protein transfer, cytoplasmic trafficking and nuclear targeting of the Vir protein/T-DNA complex (T-complex), Vir protein removal, T-DNA integration, and transgene expression. However, none of these mutants identify genes globally affecting plant transformation susceptibility.

### SUMMARY

A new method is disclosed that improves plant susceptibility to Agrobacterium-mediated transformation. This new method is easier to use and more widely applicable to many plant species that are not currently open to Agrobacterium-mediated plant transformation. Using this method, some plant species have been shown to be five- to ten-fold more susceptible to Agrobacterium-mediated transformation. This new method opens the door to how plant transformation is approached.

A myb transcription factor designated MTF was disclosed that negatively regulates plant transformation susceptibility. An integrin domain-like protein (which is under negative regulation by MTF) is involved in Agrobacterium attachment to plant cells and, thus, is a positive mediator of transformation: plants over-expressing the integrin domain-like protein are more susceptible to transformation, whereas plants mutant for the integrin domain-like protein are less susceptible. Manipulation of these elements allows improved control of Agrobacterial transformation of plants, including crops.

Agrobacterium-mediated transformation is the most widely used technique for generating transgenic plants. However, transformation remains a major limitation to enhancement of major crops through biotechnology. The first known regulator of plant transformation susceptibility is described herein. An Arabidopsis myb transcription factor (MTF) negatively regulates plant transformation susceptibility. DNA insertions in the mtf gene made Arabidopsis lines hyper-susceptible to transformation by several Agrobacterium strains. In addition, RNAi targeting of MTF in the transformation-recalcitrant Arabidopsis ecotype Bl-1 resulted in increased transformation susceptibility accompanied by increased bacterial attachment to roots.

Transcriptional profiling of wild-type and mtf mutant plants revealed down-regulation of the WRKY48 transcription factor gene in the mtf mutants. Mutation of WRKY48 resulted in hyper-susceptibility to transformation, as did over-expression of two genes that were up-regulated in the mtf mutants [At1g50060 or At5g15725]. Arabidopsis roots inoculated with Agrobacteria expressing a trans-zeatin secretion (TZS) gene showed decreased expression of MTF and a corresponding increase in transformation susceptibility.

When the Arabidopsis myb gene is overexpressed in Arabidopsis, the plants grow much larger, the roots are longer, and the leaves are darker green. This may be a useful agronomic trait if this is confirmed for crop plants grown in the field.

Myb transcription factors and integrin-like proteins, alone or in combination are useful to achieve a desired effect on transformation by manipulating Agrobacterial transformation in a plant. For example, an integrin-like protein is designated At14a, and the myb transcription factor is MTF. Increasing Agrobacterium-mediated transformation of recalcitrant species, and tissues of these species, is achieved by over-expressing of the At14a gene. In particular, some tissues that are easy to regenerate but difficult to transform may not bind Agrobacterium well, and over-expressing At14a may improve binding and transformation. Over-expression of the At14a gene produces an integrin domain-like protein in the Arabidopsis ecotype BI-1 increasing bacterial binding to roots, and also increasing root transformation. This ecotype is highly recalcitrant to Agrobacterium-mediated transformation, and binds bacteria poorly to roots.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIGS. 1A-1I. Expression of MTF influences plant susceptibility to Agrobacterium-mediated transformation. 1A Percentage of root segments developing tumors in plants inoculated with A. tumefaciens A208. 1B Representative plates showing increased transformation susceptibility of mutants. 1C Map of T-DNA insertion positions in MTF. Numbers indicate nucleotide positions; +1 indicates translation start site 1D Relative MTF transcript levels in wild-type, hat3, and mtf1-4 (previously mtf2) roots 1E Transformation susceptibility of root segments from wild-type, mtf1-4, and mtf1-4 plants complemented with a MTF cDNA. Numbers indicate individual T2 generation lines 1F Relative MTF transcript levels in roots of wild-type, mff1-4 (previously mtf2), and complemented mff1-4 (previously mtf2) lines 1, 2, 3, and 5 as in 1E 1G-1I, Down-regulation of MTF by RNAi in roots of ecotype Bl-1 increases transformation susceptibility 1G and attachment of Agrobacteria to roots 1I. Numbers indicate individual T2 generation MTF-RNAi lines and empty vector (EV) line. 1H Relative MTF transcript levels in roots of Bl-1 and lines 2, 9, 10, and EV. 1I Attachment of GFP-tagged A. tumefaciens A208 to root segments of Col-0, Bl-1 and MTF-RNAi lines 2, 8, 9, 10, and EV. Error bars in all figures indicate s.e.m. from 3 (for relative transcript levels) or 5 (for percentage of roots developing tumors) replicates.

FIGS. 2A-2C. Phytohormone pre-treatment of Arabidopsis roots increases susceptibility to Agrobacterium-mediated transformation. 2A Representative plates showing tumors on root segments from Arabidopsis ecotypes following 0, 1, and 3 days of phytohormone pre-treatment before infection with A. tumefaciens A208. 2B Percentage of root segments developing tumors. 2C Transient transformation after 3 d phytohormone pre-treatment of root segments followed by infection with A. tumefaciens At849.

FIGS. 3A-3D. A. tumefaciens tzs mutant is less virulent than the wild-type strain. 3A Percentage of root segments developing tumors after inoculation with tzs mutant and wild-type A. tumefaciens. 3B Relative MTF transcript levels in roots infected with tzs mutant and wild-type A. tumefaciens. 3C MTF promoter-EYFP construction expresses constitutively in transgenic Arabidopsis. 3D Inoculation of MTF promoter-EYFP transgenic roots with TZS and tzs mutant A. tumefaciens.

FIGS. 4A-4E. Trans-zeatin treatment increases susceptibility to Agrobacterium-mediated transformation by strains lacking TZS. 4A-4B Percentage of root segments developing tumors in Col-0 4A and Bl-1 4B inoculated with A. tumefaciens A348 and A281 in the absence or presence of trans-zeatin. Relative MTF transcript levels in root segments of Col-0 4C and Bl-1 4D treated for two days with trans-zeatin. 4E Attachment of GFP-tagged A. tumefaciens A281 to root segments of Col-0 and Bl-1 treated for 24 h with 0 or 1.4 μM trans-zeatin.

FIGS. 5A-5D. Manipulation of Arabidopsis genes that are regulated by MTF increases susceptibility to Agrobacterium-mediated transformation. 5A Percentage of root segments developing tumors in transgenic plants over-expressing At1g50060 or At5g15725 cDNAs inoculated with A. tumefaciens A208. Numbers indicate individual T2 generation lines. Relative transcript levels of At1g50060 5B and At5g15725 5C after inoculation with A. tumefaciens A208 (TZS⁺), A348 (TZS⁻), or A281 (TZS⁻). 5D Percentage of root segments developing tumors in T-DNA-disruption mutants of genes down-regulated in mtf plants.

FIGS. 6A-6D. Arabidopsis mtf mutants are resistant to Botrytis cinerea. Col-0 and mtf1-4 (previously mtf2) plants were spray 6A or drop-inoculated 6B with B. cinerea spores. Average lesion diameter 6C was calculated from drop-inoculated leaves 4 days post-inoculation. 6D Relative ORA59 transcript levels in Col-0 and mtf1-4 (previously mtf2) leaves 0, 24, and 48 h post-inoculation.

FIG. 7. Mutation of MTF increases root transformation susceptibility to multiple Agrobacterium strains. Root segments from wild-type or homozygous mtf1-4 (previously mtf2) mutant plants were inoculated with A. tumefaciens A348 or A281. The percentage of root segments that developed tumors was calculated. Error bars indicate s.e.m. from five replicates.

FIG. 8. Decreasing MTF expression increases transformation susceptibility of Arabidopsis ecotype Bl-1. Transgenic T1 generation Arabidopsis ecotype Bl-1 plants expressing a RNAi construction which targets MTF were inoculated with A. tumefaciens A208. The percentage of root segments that developed tumors was calculated. Numbers below the bars indicate individual Bl-1::MTF-RNAi lines. Error bar indicates s.e.m. from five replicates.

FIG. 9. RT-PCR analysis of transcripts of genes up-regulated in hat3 and homozygous mtf1-4 (previously mtf2) roots. Amplified fragments were fractionated by electrophoresis through agarose gels, stained with ethidium bromide, and photographed. The ACT2 gene was used as a normalization control.

FIG. 10. RT-PCR analysis of transcripts of genes down-regulated in hat3 and homozygous mtf1-4 (previously mtf2) roots. Amplified fragments were fractionated by electrophoresis through agarose gels, stained with ethidium bromide, and photographed. The ACT2 gene was used as a normalization control.

FIG. 11. Over-expression of several Arabidopsis genes that are regulated by MTF increases plant susceptibility to Agrobacterium-mediated transformation. Root segments from T1 generation transgenic plants over-expressing At2g40960, At1g50060, At5g46295, or At5g15725 cDNAs were inoculated with A. tumefaciens A208. The percentage of root segments that developed tumors was calculated. Numbers indicate individual transgenic lines. Error bar indicates s.e.m. from five replicates.

FIGS. 12A-12B. Homozygous mtf1-4 (previously mtf-2) plants show no alteration in susceptibility to Alternaia brassicicola or Pseudomonas syringae DC3000. 12A Leaves of wild-type and homozygous mtf1-4 (previously mtf2) mutant plants were inoculated with 5 μL of a 500,000 spores/mL A. brassicicola spore suspension. The leaves were photographed 5 d after inoculation. 12B Leaves of wild-type and homozygous mtf1-4 (previously mtf2) mutant plants were inoculated with wild-type and hrcC mutant Pseudomonas syringae pv. tomato. After 0 and 3 d, leaf sections were ground and the bacteria plated.

FIG. 13. MTF-RNAi lines in Arabidopsis ecotype BI-1 show varying levels of MTF transcripts.

FIG. 14. Decreasing MTF transcripts in the transformation-recalcitrant Arabidopsis ecotype BI-1 increases susceptibility to Agrobacterium. Bacterial concentration (10⁸ cfu/mL).

FIG. 15. MTF-RNAi lines show increased attachment of GFP-labeled Agrobacteria.

FIG. 16. Arabidopsis and crop myb transcription factors are highly homologous. FIG. 16 discloses SEQ ID NOS 71-75, respectively, in order of appearance.

FIG. 17. Expression of the rice MTF ortholog in the Arabidopsis mtf1-4 (previously mtf2) mutant results in lower transformation susceptibility.

FIG. 18. A rice MTF-RNAi line shows increased transient transformation.

FIGS. 19A-19B. Expression of the Brassica oleracea MTF ortholog in the Arabidopsis mtf1-4 (previously mtf2) mutant results in lower transformation susceptibility. 19A shows results of a transient GUS assay; 19B a root tumorigenesis assay.

FIGS. 20A-20B. Expression of the Brassica napus MTF ortholog in the Arabidopsis mtf1-4 (previously mtf2) mutant results in lower transformation susceptibility. 20A shows results of a transient GUS assay; 20B a root tumorigenesis assay.

FIG. 21. Expression of the Brassica rapa MTF ortholog in the Arabidopsis mtf1-4 (previously mtf2) mutant results in lower transformation susceptibility. (Transient GUS assay).

FIGS. 22A-22E. MTF Sequences: Double underlined nucleotides indicate start codons; single underlined nucleotides indicate stop codons; italic bold nucleotides indicate part of the 5′- and 3′ untranslated sequences on the cDNA clones: 22A Arabidopis MTF and MTF (SEQ ID NOS 76 and 74, respectively, in order of appearance); 22B-22E orthology sequences [Rice, Brassica napus, Brassica rapa, Brassica oleracea] (SEQ ID NOS 77, 75; 78, 71; 79, residues 1-233 of SEQ ID NO: 72; 80 and 73, respectively, in order of appearance).

FIG. 23. At14a: The mff1-4 (previously mtf2) mutant shows increased At14a transcript levels; At14a was of interest because its expression is up-regulated in the Arabidopsis mtf myb transcription factor mutant; this mutant is hyper-susceptible to Agrobacterium-mediated transformation.

FIG. 24. At14a Sequences: The Arabidopsis data bases indicate that there are two identical At14a gene sequences (“At3G28290” and “At3G28300” both disclosed as SEQ ID NO: 81), plus two related sequences (SEQ ID NOS 82-83, respectively, in order of appearance).

FIGS. 25A-25B. At14a Transformation: The transformation susceptibility of the Arabidopsis At14a mutant is lower than that of wild-type Col-0 plants. 25A shows a transient GUS assay; 25B antibiotic resistant calli.

FIG. 26. At14a Binding: Arabidopsis At14a shows decreased binding of GFP-labeled A. tumefaciens A348.

FIG. 27. At14a Binding: Arabidopsis At14a mutant shows decreased binding of GFP-labeled A. tumefaciens A208.

FIG. 28. At14a and mtf1-4: The At14a mutant shows decreased binding, and the mtf1-4 (previously mtf2) shows increased binding, of A. tumefaciens compared to Arabidopsis Col-0 (using scanning electron microscopy of unlabeled Agrobacteria).

FIG. 29. Arabidopsis plants infected with TZS and/or iP-producing A. tumefaciens strains show lower amounts of MTF transcripts.

FIGS. 30A-30C. Arabidopsis plants infected with an A. tumefaciens miaA mutant show decreased transformation susceptibility. 30A binding; 30B shows a transient GUS assay; 30C shows kanamycin resistant calli.

FIG. 31 Amino Acid Sequences of the Arabidopsis and Rice MTF1 proteins. The T-DNA insertion that disrupts the Arabidopsis gene occurs at the position of the triangle; therefore, everything following that position is no longer in the mutant protein; consequently, mutations in the rice gene were made at positions approximately at this Q (glutamine) amino acid to disrupt the downstream sequence. FIG. 31 discloses SEQ ID NOS 74 and 75, respectively, in order of appearance.

FIG. 32 Nucleic Acid and Amino Acid Sequences of the Carboxy-terminal Region of the Rice MTF1 Protein (Os05g0412000). Sequences of sgRNA guides are highlighted in blue; the PAM sequences are in red; note that the sgRNAs were designed to work in pairs such that the C-terminal region of the protein would be deleted; the glutamine (Q) that is the site of disruption (a marker) in the Arabidopsis mtf1-4 mutant is encoded starting at nucleotide #713. FIG. 32 discloses the nucleotide sequence as SEQ ID NO: 88, coding frame 1 amino acid sequence as SEQ ID NO: 89, coding frame 2 amino acid sequences as SEQ ID NOS 90 and 91, respectively, in order of appearance, and coding frame 3 amino acid sequence as SEQ ID NO: 92.

FIG. 33 T-DNA Binary Vector map of the T-DNA binary vector used to transform mtf1 mutant rice line to assess transformation susceptibility; the most important feature is the gusA-intron gene that allows transient GUS activity to be measured using the chromogenic reagent X-gluc; the experimental protocol used to test wild-type and mtf1 mutant rice lines for transformation susceptibility;

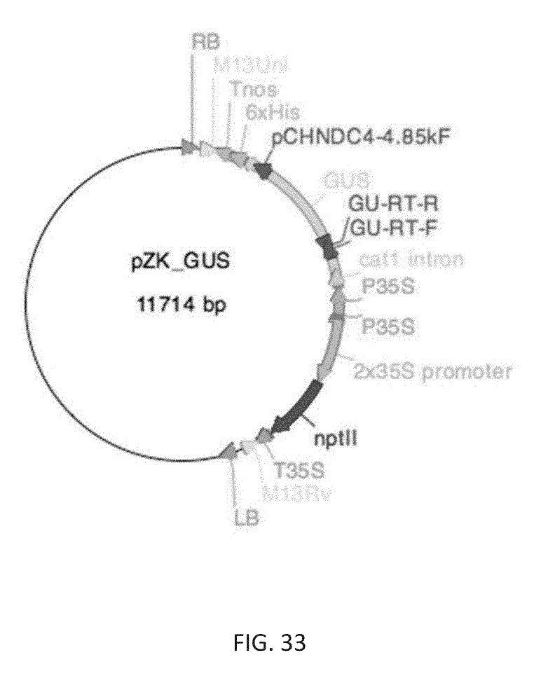

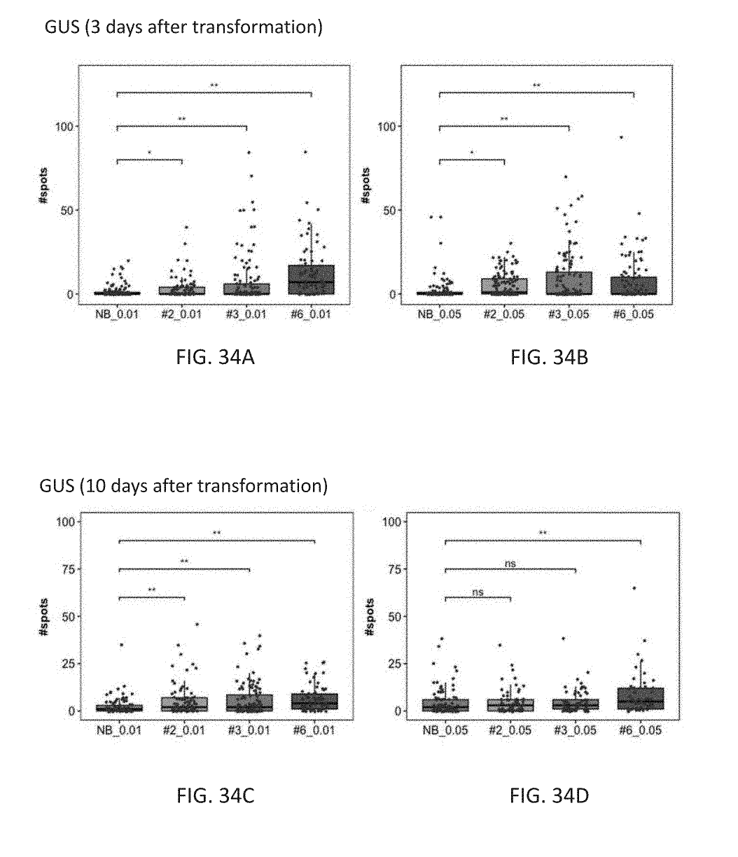

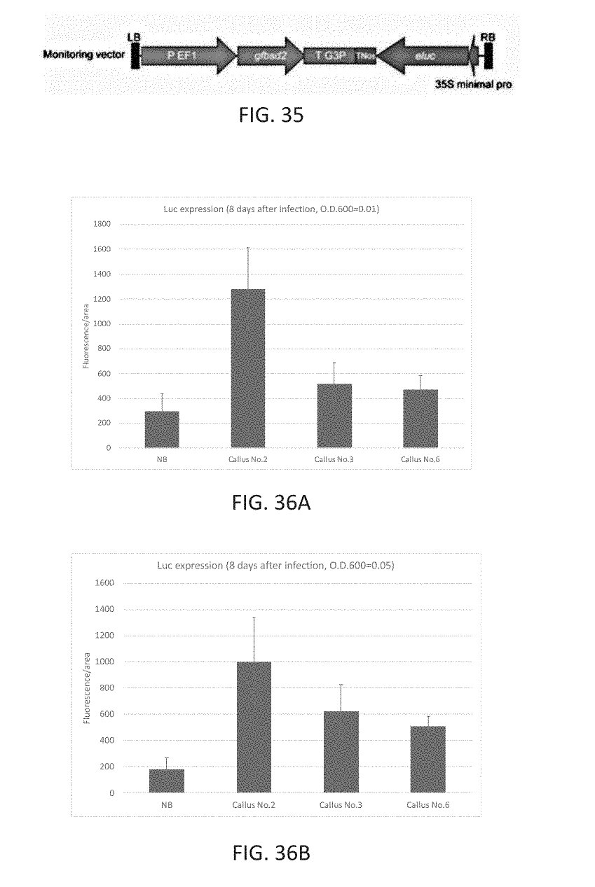

pZK_gMyb#3,5_PubiMMCas9-10-19
2017 Nov. 21 Put 6 seeds on N6D medium
December 12, December 24 Transfer calli to new N6D médium
2017 Dec. 25 Genotyping
2018 Jan. 7 Transform pZK_GUS to NB, Myb mutants
2018 Jan. 10 Wash Agro, GUS assay (Day3), start NPTII selection
2018 Jan. 17 GUS assay (Day10)
#2 27 bp del.=in frame 9 a.a. (HHHPPQIGH (SEQ ID NO: 84)) del. homo
#3 171 bp del.=in frame 57 a.a. (IGHF . . . AAGMA (SEQ ID NOS 85 and 86, respectively)) del. homo
#6 24 bp del.=in frame 8 a.a. (PPQIGHFH (SEQ ID NO: 87))del. homo
   additionally, the extent of the deletions in the MTF1 gene are noted in the various CRISPR-generated mutants. FIG. 33 discloses “6×His” as SEQ ID NO: 93.

FIG. 34(A-D) Results of the Transient Transformation Assay in which the number of blue X-gluc staining spots are counted for 100 calli of each rice genotype (NB=Nipponbare wild-type, the mutant numbers match the deletions noted below); the assays were conducted 3 (FIGS. A, B) and 10 days (FIG. C, D) after infection, using Agrobacterium at a concentration of 10⁷ cfu/ml (0.01) (FIG. 34 A, C) or 5×10⁷ cfu/ml (0.05) (FIG. 34 B, D); #2 27 bp del.=in frame 9 a.a. (HHHPPQIGH (SEQ ID NO: 84)) del. homo; #3 171 bp del.=in frame 57 a.a. (IGHF . . . AAGMA (SEQ ID NOS 85 and 86, respectively)) del. homo; #6 24 bp del.=in frame 8 a.a. (PPQIGHFH (SEQ ID NO: 87))del. homo.

FIG. 35 Vector to Detect Transformation. map of the monitoring vector to detect stable transformation of wild-type and mtf1 mutant rice calli; the important feature of this vector is a click beetle luciferase gene (eluc) with minimal CaMV 35 S promoter; this promoter cannot function to transcribe the eluc gene unless the T-DNA has integrated into the rice genome near a transcriptional enhancer; thus, this vector measures stable transformation (T-DNA integration into the genome;

pZK_gMyb#3,5_PubiMMCas9-10-19
2017 Nov. 21 Put 6 seeds on N6D medium
December 12, December 24 Transfer calli to new N6D médium
2017 Dec. 25 Genotyping
2018 Jan. 14 Transform Monitoring vector to NB, Myb mutants
2018 Jan. 17 Wash Agro, start blasticidin S selection
2018 Jan. 18 Luc assay (Day4)<-Luc signal was too weak
2018 Jan. 22 Luc assay (Day8)
#2 27 bp del.=in frame 9 a.a. (HHHPPQIGH (SEQ ID NO: 84)) del. homo
#3 171 bp del.=in frame 57 a.a. (IGHF AAGMA (SEQ ID NOS 85 and 86, respectively)) del. homo
#6 24 bp del.=in frame 8 a.a. (PPQIGHFH (SEQ ID NO: 87))del. homo
   is the protocol used for this experiment, and a description of the various rice mtf1 mutants used.

FIG. 36(A-B) Stable Transformation. Stable transformation results of wild-type (NB) and mtf1 mutant rice calli using the monitoring vector of FIG. 35; rice calli (100/line) were infected with Agrobacterium, and luciferase fluorescence was quantified after 8 days. (FIG. 36A left), calli were infected with Agrobacterium at 10⁷ cfu/ml; (FIG. 36B right), calli were infected with Agrobacterium at 5×10⁷ cfu/ml.

### DETAILED DESCRIPTION

### Arabidopsis and Rice myb Transcription Factors are Highly Homologous

A genetic screen for Arabidopsis mutants displaying a hyper-susceptible to Agrobacterium transformation (hat) phenotype was performed. The gene disrupted in the hat3 mutant encodes a putative myb-family transcription factor (MTF) that negatively regulates susceptibility to Agrobacterium-mediated transformation. Over-expression of an integrin-like protein results in plants that are hyper-susceptible to transformation. Manipulation of MTF, members of this protein family, and members of the integrin domain-like protein family, for example At14a allows improved control of Agrobacterium transformation, including in crops.

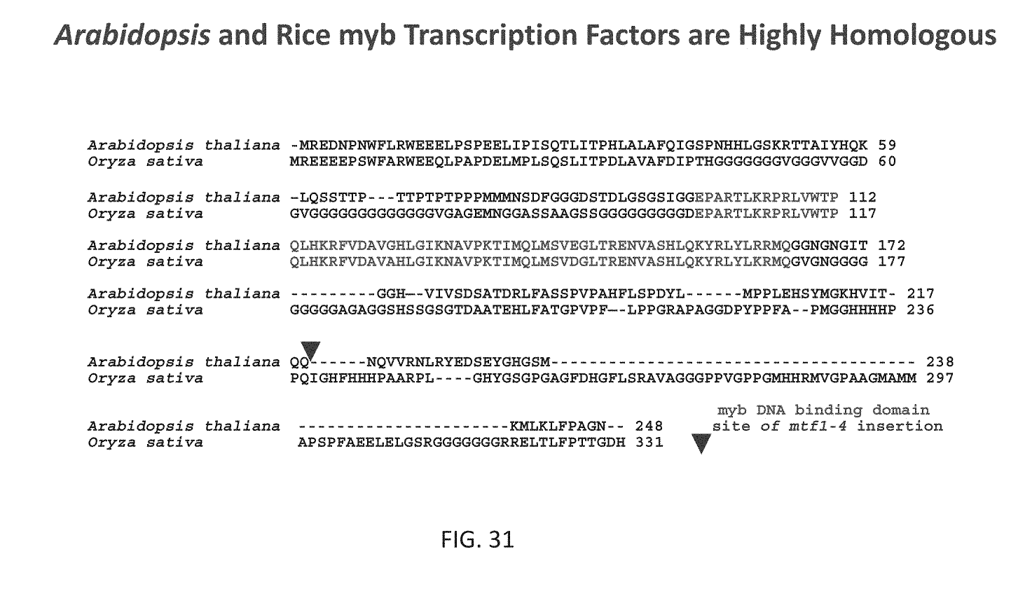

Amino acid sequences of the Arabidopsis and rice MTF1 proteins are shown in FIG. 31. The T-DNA insertion that disrupts the Arabidopsis gene occurs at the position of the triangle; therefore, everything following that position is no longer in the mutant protein. Consequently, mutations in the rice gene were made at positions approximately at this Q (glutamine) amino acid that would disrupt the downstream sequence.

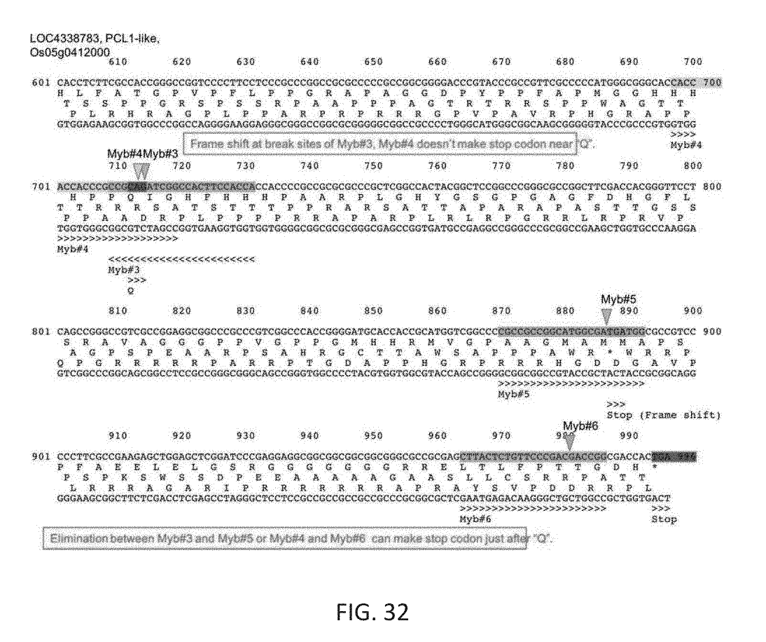

The nucleic acid and amino acid sequence of the carboxy-terminal region of the rice MTF1 protein (0505g0412000) is shown in FIG. 32. Sequences of sgRNA guides are highlighted in blue; the PAM sequences are in red. The sgRNAs were designed to work in pairs such that the C-terminal region of the protein would be deleted. The glutamine (Q) that is the site of disruption (a marker) in the Arabidopsis mtf1-4 mutant is encoded starting at nucleotide #713.

FIG. 33 is a map of the T-DNA binary vector used to transform mtf1 mutant rice lines to assess transformation susceptibility; the most important feature is the gusA-intron gene that allows transient GUS activity to be measured using the chromogenic reagent X-gluc.

FIG. 33B is the experimental protocol used to test wild-type and mtf1 mutant rice lines for transformation susceptibility; additionally, the extent of the deletions in the MTF1 gene are noted in the various CRISPR-generated mutants.

Results of the transient transformation assay in which the number of blue X-gluc staining spots are counted for 100 calli of each rice genotype are shown in FIG. 34(A-D) (NB=Nipponbare wild-type, the mutant numbers match the deletions noted below); the assays were conducted 3 and 10 days after infection, using Agrobacterium at a concentration of 10⁷ cfu/ml (0.01; A, C) or 5×10⁷ cfu/ml (0.05; B-D). #2 27 bp del.=in frame 9 a.a. (HHHPPQIGH (SEQ ID NO: 84)) del. homo; #3 171 bp del.=in frame 57 a.a. (IGHF . . . AAGMA (SEQ ID NOS 85 and 86, respectively)) del. homo; #6 24 bp del.=in frame 8 a.a. (PPQIGHFH (SEQ ID NO: 87))del. homo.

A map of the monitoring vector to detect stable transformation of wild-type and mtf1 mutant rice calli is in FIG. 35 (A-B). The important feature of this vector is a click beetle luciferase gene (eluc) with a minimal CaMV35 S promoter. This promoter cannot function to transcribe the eluc gene unless the T-DNA has integrated into the rice genome near a transcriptional enhancer; thus, this vector measures stable transformation T-DNA integration into the genome. FIG. 35B is the protocol used for this experiment, and a description of the various rice mtf1 mutants used.

Stable transformation results of wild-type (NB) and mtf1 mutant rice calli using the monitoring vector or shown in FIG. 36. Rice calli (100/line) were infected with Agrobacterium, and luciferase fluorescence was quantified after 8 days. Calli were infected with Agrobacterium at 10⁷ cfu/ml; t), or with Agrobacterium at 5×10⁷ cfu/ml.

### Identification and Characterization of mtf Mutants

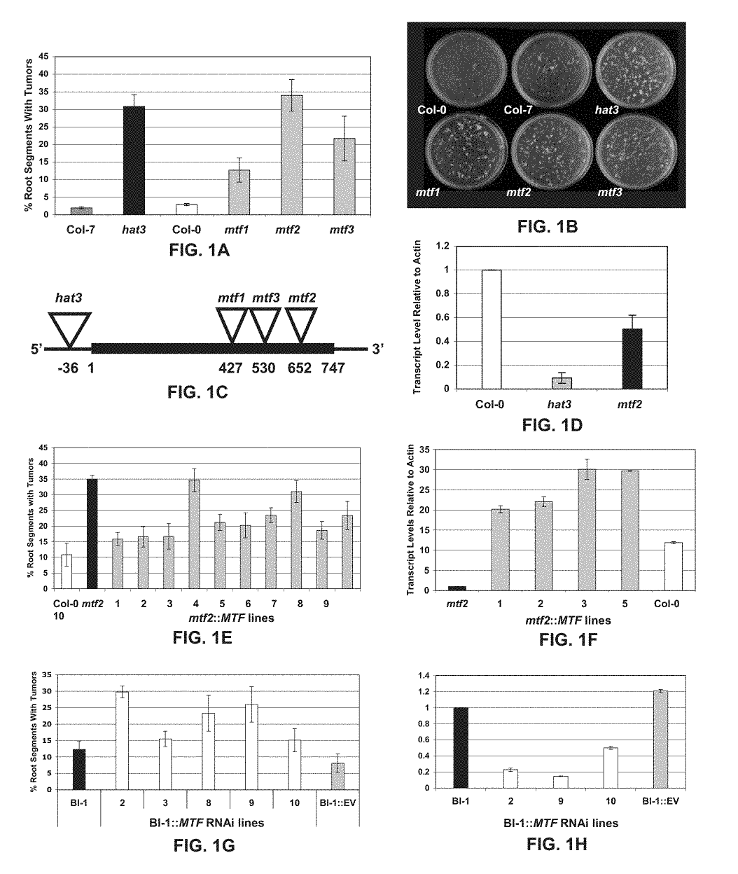

To identify mutants with increased susceptibility to Agrobacterium-mediated transformation, ˜4000 mutagenized plants were screened from an Arabidopsis T-DNA activation-tagged library (Weigel, 2000). The mutant hat3 displayed a ˜10-fold increase in transformation susceptibility (FIG. 1A, B). TAIL-PCR (Liu et al., 1995) was used to identify the T-DNA/plant junction in hat3, and it was discovered that the T-DNA had inserted into the 5′ untranslated region of a putative myb transcription factor (MTF) gene, At2g40970, 36 bp upstream of the start codon (FIG. 1C). MTF has a single Myb DNA-binding domain of the SHAQKYF (SEQ ID NO: 1) type that is unique to plants, and is a member of a five-gene family (Hazen et al., 2005). The DNA-binding domain is similar to those found in proteins associated with two-component signal transduction systems (Hwang et al., 2002), the B-type Arabidopsis response regulators (ARRs), GOLDEN2-LIKE (GLK), and PRR2 (Hazen et al, 2005).

Homozygous mutant plants were not recoverable from self-fertilized progeny of hat3, suggesting that complete disruption of MTF may be lethal. Self-fertilization of three additional T-DNA MTF insertion mutants, SALK_072082 (mtf1), SALK_072083 (mtf1-4), and SALK_102624 (mtf3), resulted in a homozygous mutant only for mtf1-4 (previously mtf2). The insertion in mtf1-4 (previously mtf2) permitted expression of ˜85% of the MTF open reading frame, indicating that the majority of MTF protein is essential for Arabidopsis viability. Homozygous mtf1-4 (previously mtf2) plants showed an ˜11-fold increase in transformation susceptibility. Heterozygous mtf1 and mtf3 mutants displayed 4-7-fold increased transformation susceptibility (FIG. 1A, B). Thus, all four mtf mutant lines displayed a hat phenotype, highlighting the importance of MTF in transformation. Quantitative real-time RT-PCR assays revealed that MTF transcript levels decreased 2-fold in mtf1-4 (previously mtf2) and >12-fold in hat3 (FIG. 1d), demonstrating that transformation susceptibility negatively correlates with MTF transcript levels.

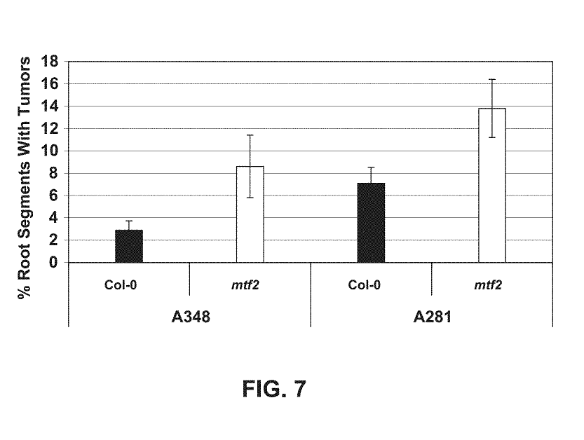

The transformation experiments described herein were carried out using A. tumefaciens A208 that contains a nopaline-type of Ti plasmid. Commonly used Agrobacterium strains were, for example A208, A348, A281 (Zhu et al., 2003; and Nam et al., 1999). To assess whether mtf1-4 (previously mtf2) shows increased susceptibility to other A. tumefaciens strains, root transformation assays were conducted using the octopine-type strain A348 and the succinamopine-type strain A281. The mtf1-4 (previously mtf2) mutant displayed 2-3-fold increased transformation susceptibility to these strains (FIG. 7). Thus, MTF plays an important role in plant susceptibility to different Agrobacterium strains.

Further studies used homozygous mtf1-4 (previously mtf2) plants. Ectopic expression of the MTF cDNA in mtf1-4 (previously mtf2) resulted in several transgenic lines with restored levels of wild-type susceptibility to Agrobacterium-mediated transformation (FIG. 1E). These transgenic lines individually expressed various levels of MTF mRNA (FIG. 1F). Complementation experiments confirm that disruption of the MTF gene is responsible for increased transformation susceptibility.

The mtf1-4 (previously mtf2) mutant is hyper-susceptible to different strains of A. tumefaciens carrying nopaline-, octopine-, and succinomanopine-type Ti plasmids, indicating that MTF is a negative regulator of Agrobacterium-mediated transformation. Transformation recalcitrance of some Arabidopsis ecotypes results from decreased binding of Agrobacterium to roots. Other ecotypes are debilitated in T-DNA integration, a late stage of transformation (Nam et al., 1997). Reducing MTF expression in Bl-1, a highly recalcitrant ecotype, increased transformation susceptibility and bacterial attachment, highlighting the potential to increase transformation susceptibility of recalcitrant plant species by down-regulating expression of MTF orthologs.

The importance of phytohormones in increasing transformation prompted investigation of the role of cytokinins in transformation. Agrobacterium strains containing nopaline-type Ti plasmids secrete trans-zeatin, mediated by the vir region-localized gene TZS. A. tumefaciens tzs mutants are less virulent than are TZS strains. The presence of TZS on the bacterial surface (Aly et al, 2008) may mean that metabolites from wounded plant cells may be converted into trans-zeatin at infection sites, resulting in down-regulation of MTF and consequent increased transformation susceptibility. Indeed, exogenous application of kinetin during infection increased the susceptibility of Arabidopsis roots infected with an Agrobacterium tzs mutant (Hwang et al., 2010). Down-regulation of MTF expression by cytokinins provides a molecular explanation for the importance of TZS to Agrobacterium-mediated transformation (Zhan et al, 1990). Although influential, cytokinin signaling is not essential for Agrobacterium-mediated transformation because many virulent Agrobacterium strains do not secrete cytokinins.

Regulation of gene expression by MTF is highly specific. Fewer than 40 genes are significantly up- or down-regulated 1.5-fold in the mtf mutants. One of the up-regulated genes, At1g50060 encoding a basic PR1-like protein, increased transformation susceptibility when over-expressed in Arabidopsis. Unlike its acidic counterpart, PR-1, At1g50060 is not salicylic acid (SA)-responsive, pathogen-induced, nor is its expression correlated with the establishment of systemic acquired resistance (Niki et al., 1998). However, At1g50060 transcripts are negatively regulated by a variety of biotic and abiotic stresses (Zimmerman et al., 2004). Thus, At1g50060 does not encode a defense-related protein. Increased transformation susceptibility of the wrky48 mutant suggests that Agrobacterium manipulates host defense responses to its advantage. Previously Veena et al. (2003) showed that infection of plant cells by transfer-competent Agrobacterium strains suppresses host defense gene expression 30-36 h after infection, although these genes are induced as early as 3-12 h after infection (Veena et al., 2003). MTF is a specific regulator of plant susceptibility to Agrobacterium as evidenced by lack of increased susceptibility to A. brassicicola and P. syringae. Increased resistance of the mtf mutant to Botrytis is likely due to downstream responses to decreased MTF expression.

In conclusion, MTF was identified as the first known regulator of plant susceptibility to Agrobacterium-mediated transformation. MTF regulates at least three genes independently capable of increasing transformation susceptibility. MTF also affects Agrobacterium binding to roots and integrates cytokinin secretion by Agrobacterium with transformation susceptibility. These findings pave the way for identifying orthologs of MTF in transformation-recalcitrant plant species and manipulating these genes to increase transformation efficiency of economically important crops.

### EXAMPLES

Examples are provided for illustrative purposes and are not intended to limit the scope of the disclosure.

### Example 1: Decreased MTF Expression in Arabidopsis Ecotypes Increases Transformation Susceptibility

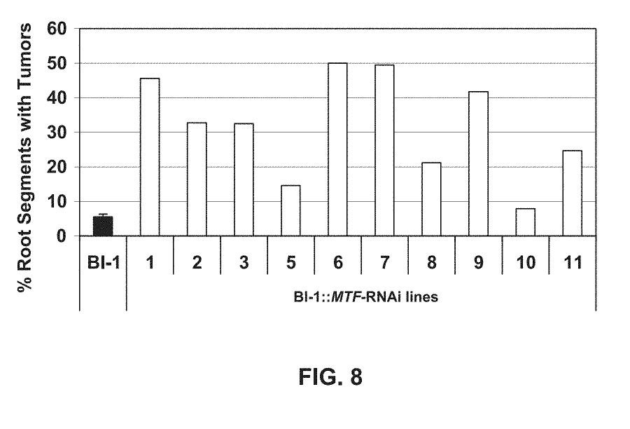

The hat3 and mtf1-4 (previously mtf2) mutants are in the Columbia background, an ecotype relatively amenable to root transformation. Arabidopsis ecotype Bl-1 is highly recalcitrant to root transformation (Nam et al., 1997), but can be transformed using a floral dip method (Mysore et al., 2000). MTF genes of ecotypes Columbia and Bl-1 are identical. An RNAi expression construction targeting MTF transcripts was introduced into ecotype Bl-1 and the derived transgenic lines were screened for root transformation susceptibility. Eight of the 10 tested T1 generation transgenic plants exhibited increased susceptibility (FIG. 8). 25 T2 generation plants from each of five MTF-RNAi lines were tested, along with a RNAi empty vector line. Three of these transgenic lines continued to show higher transformation susceptibility (FIG. 1G). RNAi lines 2 and 9, that had increased transformation susceptibility, showed 4.6- and 7-fold decreases in MTF transcripts, respectively, whereas line 10, that did not have altered susceptibility, showed only a 2-fold decrease in MTF transcript levels (FIG. 1H). A transgenic line containing an empty RNAi vector did not display altered transformation susceptibility or altered MTF transcript levels. These results indicate that transformation susceptibility of Bl-1 plants is dependent on the level of MTF transcripts.

Earlier studies indicated that roots of ecotype Bl-1 do not bind Agrobacteria well. A. tumefaciens expressing GFP showed increased bacterial attachment in the high-transforming transgenic Bl-1 RNAi lines 2 and 9 compared to that of the low-transforming line 10, the empty RNAi vector line, and wild-type Bl-1 (FIG. 1I), suggesting that decreased MTF transcripts in Bl-1 increase susceptibility during the early attachment stage of the transformation process.

### Example 2: Phytohormone Treatment Increases Transformation Susceptibility

Chateau et al. (2000) reported that phytohormone preincubation of Arabidopsis petioles increases transformation susceptibility, and hormone pre-treatment is part of the protocol to generate transgenic Arabidopsis plants from roots (Valvekens et al., 1988). Because phytohormone pretreatment of Arabidopsis root segments may enhance transformation susceptibility, which may be important in light of the fact that nopaline-type Agrobacterium strains express a trans-zeatin secretion (TZS) gene, and thus secrete cytokinins into the medium.

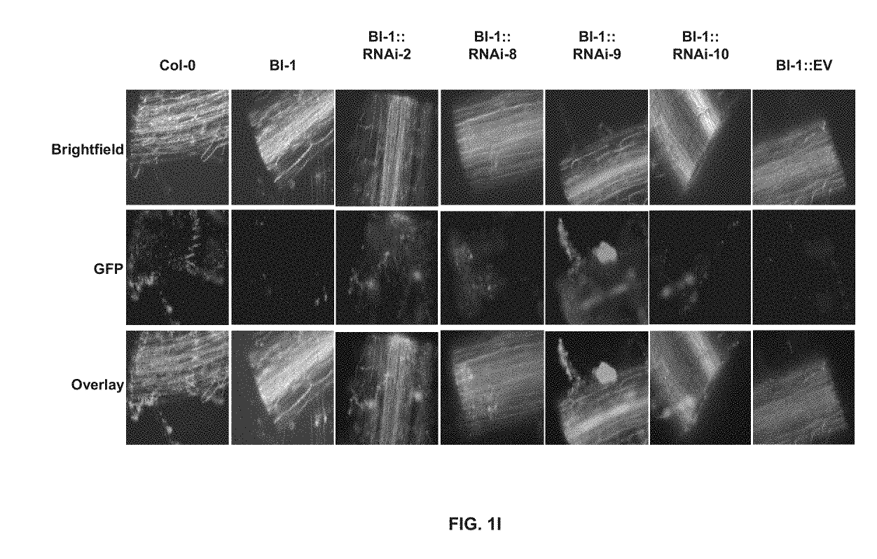

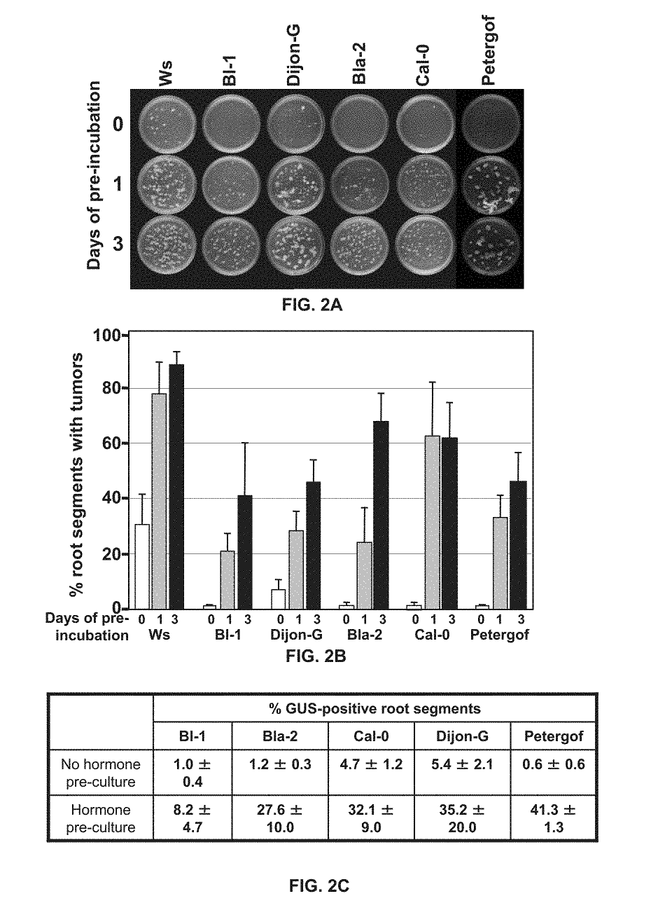

Root segments from five transformation-recalcitrant Arabidopsis ecotypes (Bl-1, Bla-2, Cal-0, Dijon-G, and Petergof) and a transformation-susceptible ecotype (Ws-2) were incubated on callus inducing medium (CIM) containing phytohormones prior to infection by Agrobacterium and scored for transformation susceptibility. All ecotypes displayed increased transformation susceptibility after one day of phytohormone pre-treatment (FIG. 2A, B). There was a further increase in transformation frequency after three days of phytohormone pre-treatment.

Whether phytohormone pre-treatment of Arabidopsis roots enhances the frequency of transient transformation was investigated, a process that does not require T-DNA integration into the plant genome. β-glucuronidase (GUS) activity, resulting from the transfer of a gusA-intron gene from Agrobacterium to plants, is a standard assay for transient transformation (Narasimhulu et al., 1996). Hormone pre-treatment of roots also increased transient transformation (FIG. 2C). Petiole explants of Arabidopsis treated with phytohormones before Agrobacterium infection showed actively dividing and dedifferentiated cells, and increased transformation efficiency. Increased DNA duplication and cell division of phytohormone treated Petunia hybrida cells correlated with increased Agrobacterium-mediated transformation (Villemont et al., 1997). Thus, phytohormone treatment sensitizes roots to Agrobacterium-mediated transformation at an early step (prior to T-DNA integration) of the transformation process.

### Example 3: MTF Expression is Repressed by Cytokinins from Agrobacterium

Ti-plasmids of some nopaline-type Agrobacterium strains carry a TZS gene that directs synthesis and secretion of cytokinins (Regier et al., 1982; Beaty et al., 1986; and Powell et al., 1988). TZS promotes transformation both by nopaline-type A. tumefaciens strains and, when transferred to strain 1855, A. rhizogenes strains. A. tumefaciens strains harboring nopaline-type Ti plasmids secrete trans-zeatin or trans-zeatin ribosides into the medium in amounts >1 μg/L (Claeys et al., 1978; McCloskey et al, 1980).

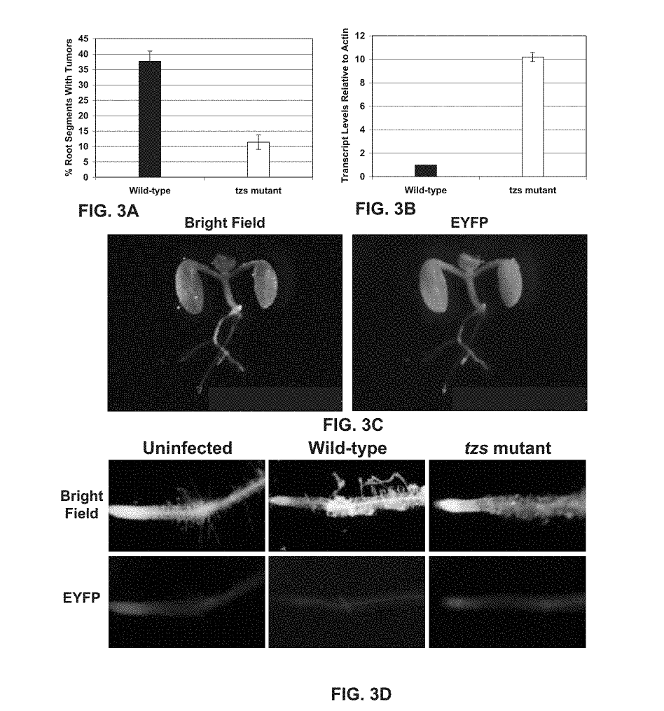

Tumorigenesis assays were conducted on Arabidopsis roots infected with the TZS⁺ strain A. tumefaciens NT1RE(pJK270) and the tzs frameshift mutant NT1RE(pJK270tzs-fs). Arabidopsis root segments infected with the tzs mutant developed fewer tumors than did roots infected with the wild-type strain (FIG. 3A). Root segments infected with wild-type bacteria had 10-fold fewer MTF transcripts than did roots infected with tzs-mutant bacteria (FIG. 3B). These results indicate that MTF is down-regulated by trans-zeatin produced by A. tumefaciens, leading to altered transformation susceptibility.

### Example 4: TZS-Expressing Agrobacteria Repress Expression of MTF

Decreased MTF transcript levels in roots co-cultivated with TZS⁺ A. tumefaciens suggests an early involvement of trans-zeatin and MTF in transformation. To determine in which root tissues this decrease in MTF expression was most pronounced, transgenic Arabidopsis lines expressing EYFP under control of the MTF promoter were generated. MTF promoter activity was constitutive in all examined plant tissues (FIG. 3C). The highly-expressing line Col7-P_{MTF}-EYFP4 was used to assess whether root tissues exhibited altered MTF expression when infected with a TZS⁺ A. tumefaciens strain. Fluorescence decreased in roots by 48 h of co-cultivation, most noticeably in the epidermal and cortical cells of the elongation zone, the region most susceptible to transformation (FIG. 3D). This decrease in fluorescence was not observed in roots incubated with the tzs frameshift mutant. These results are consistent with the decreased MTF transcript levels observed in roots co-cultivated with TZS⁺ bacteria.

### Example 5: MiaA-Expressing Agrobacteria Repress Expression of MTF and are More Susceptible to Transformation

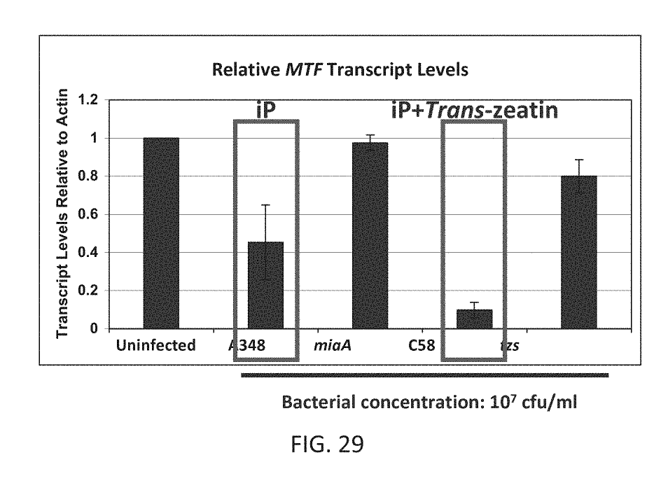

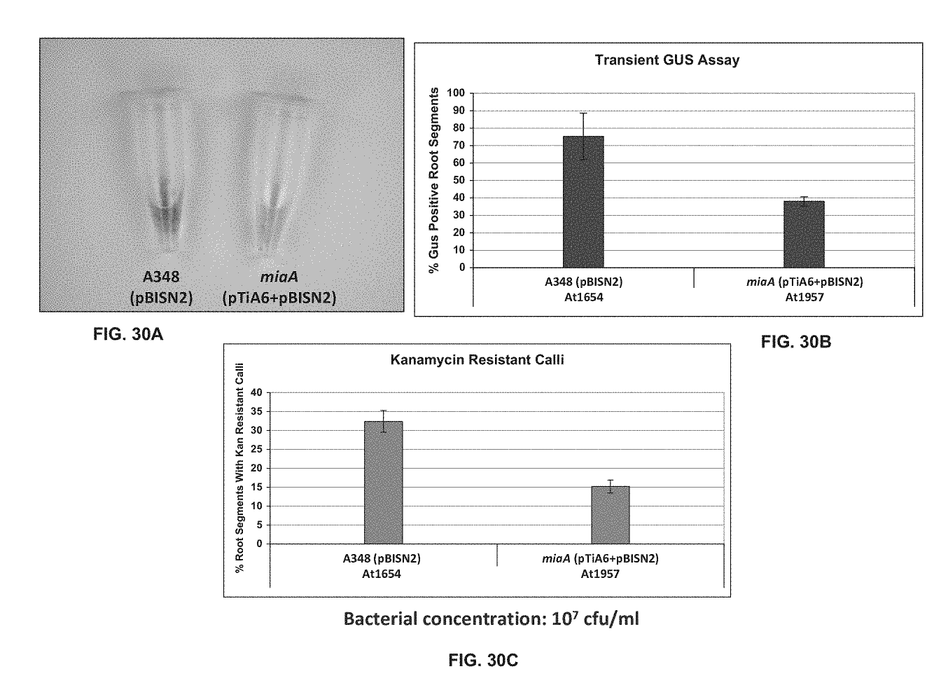

MiaA encodes an tRNA-isopentenyltransferase that isopentenylates adenine residues in tRNAs. Breakdown of tRNAs can release isopentenyladenine, a cytokinin. When Arabidopsis root segments are inoculated with Agrobacteria that contain a wild-type MIAA gene, the accumulation of MTF transcripts is repressed (FIG. 29). miaA mutant bacteria are less virulent than are wild-type bacteria (FIG. 30).

### Example 6: Cytokinin Enhances Attachment of TZS-Lacking A. tumefaciens Strains

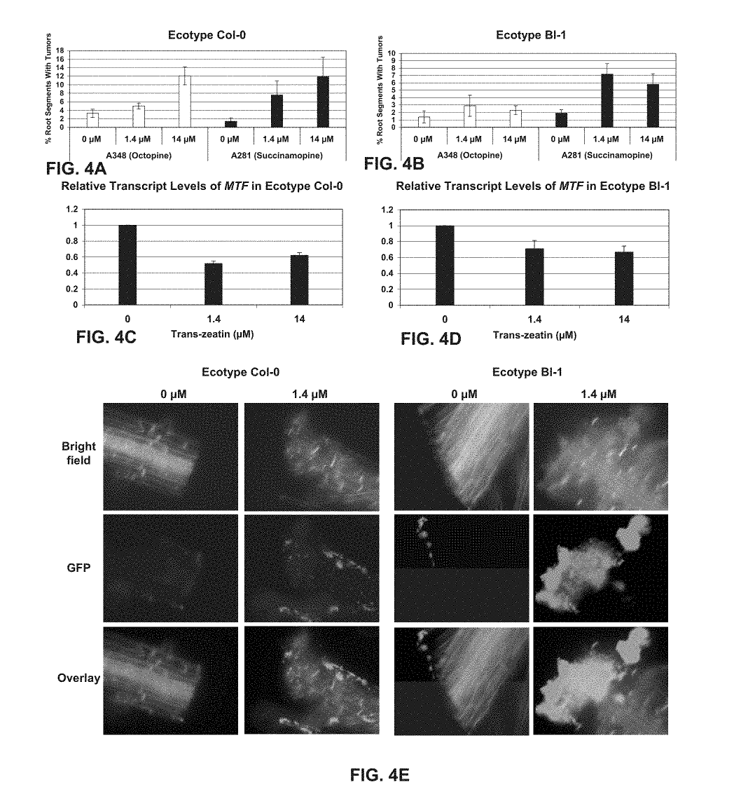

To determine whether exogenous application of trans-zeatin to roots could influence transformation susceptibility. Arabidopsis roots were incubated on medium containing trans-zeatin and they were infected with A. tumefaciens A348 or A281. Neither of these strains harbors TZS. Trans-zeatin concentrations representing the range secreted by nopaline-type A. tumefaciens strains were used. Trans-zeatin treatment of Col-0 roots resulted in a 4-8-fold increase in transformation efficiency by these A. tumefaciens strains. Ecotype Bl-1 roots infected with these strains showed a 2-3-fold increase in susceptibility (FIG. 4A, B). Incubation of roots on trans-zeatin decreased MTF transcript levels by 30-60% and also increased attachment of A. tumefaciens A281 (FIG. 4C, D, E).

### Example 7: Decreased MTF Expression Alters Expression of Genes Important for Agrobacterium-Mediated Transformation

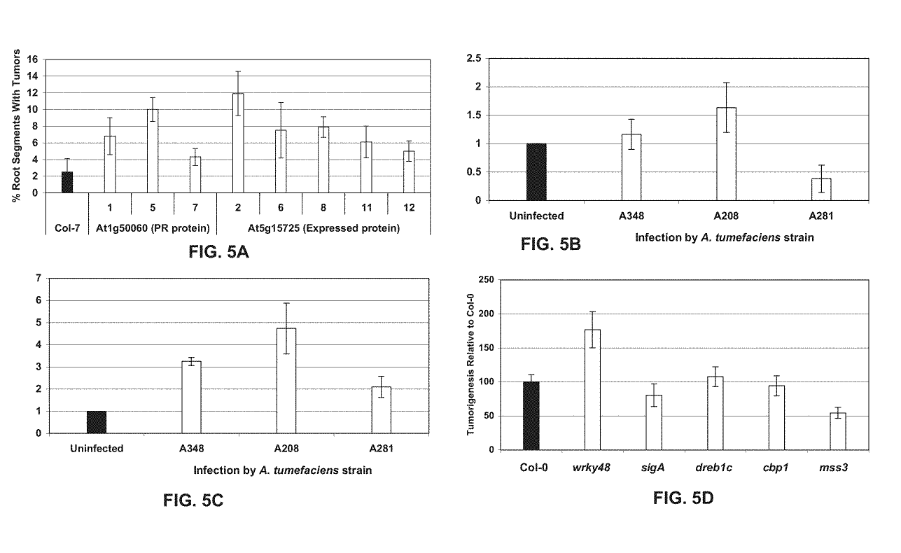

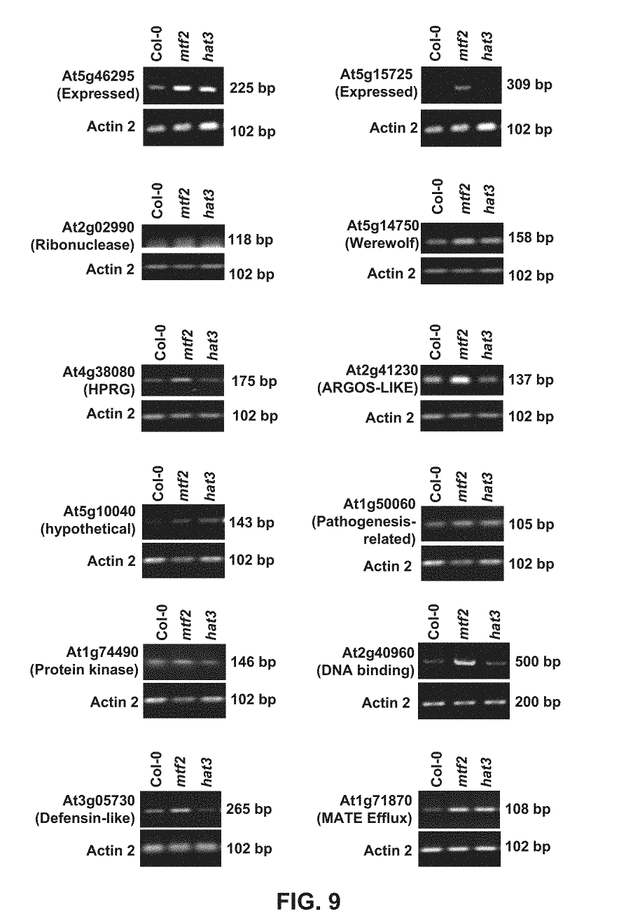

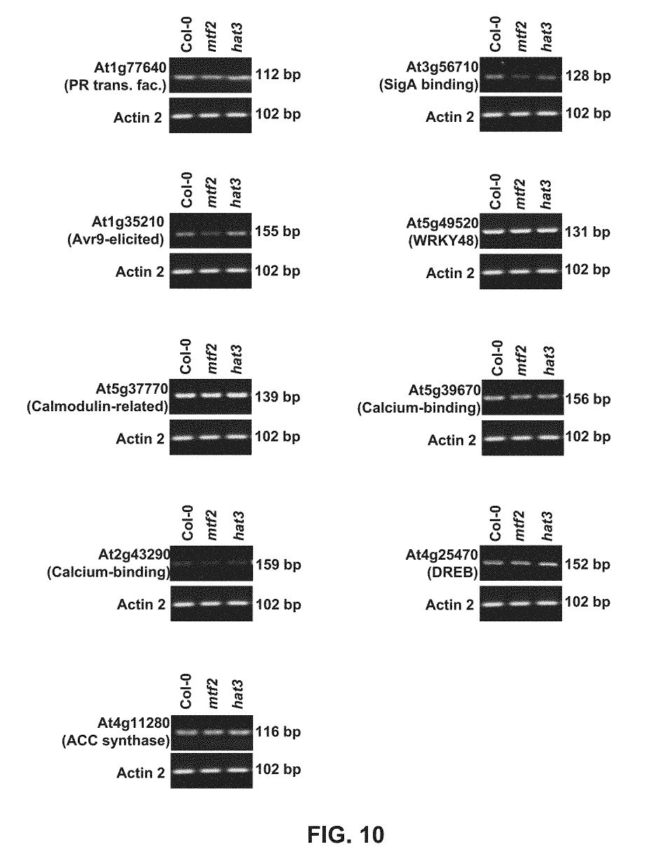

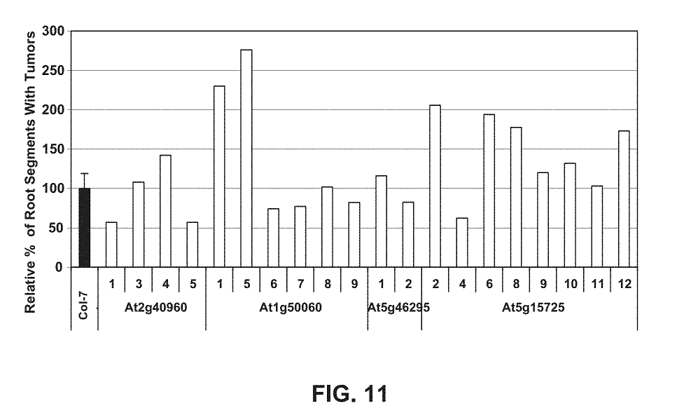

The Arabidopsis ATH1 Genome Arrays were used to identify genes whose expression is altered in wild-type, heterozygous hat3, and homozygous mtf1-4 (previously mtf2) Arabidopsis roots. A total of 39 genes exhibited statistically significant differential expression between both mtf mutants and the wild-type, and had a difference greater than 1.5-fold (Table 1). Of these, 23 genes were commonly up-regulated and 16 genes were commonly down-regulated in both mtf mutants compared to the wild-type. These results were validated using RT-PCR (FIGS. 9 and 10). cDNAs of four genes At2g40960, At1g50060, At5g46295, At5g15725 that were up-regulated in both mtf mutants were overexpressed. Transgenic T1 lines over-expressing At2g40960 and At5g46295 did not exhibit a hat phenotype (FIG. 11). However, several T1 lines over-expressing At1g50060 and At5g15725 showed increased transformation susceptibility that carried over to the T2 generation (FIG. 5A). At1g50060 is a putative pathogenesis-related 1 (PR-1)-like protein proposed to be a serine protease involved in various signaling processes (Fernandez et al, 1997; Milne et al., 2003). At1g50060 transcript levels in root segments infected with A. tumefaciens A208 (TZS⁺) and strains A348 and A281 were assessed (TZS⁻) and observed increased transcript levels only in A208-infected roots (FIG. 5B). Presumably, cytokinins produced by A. tumefaciens A208 regulate expression of MTF in the roots, which in turn regulate expression of At1g50060.

At5g15725 is annotated as an unknown protein (Tair; http://www.arabidopsis.org/). Arabidopsis root segments were infected with A. tumefaciens strains A348, A208, or A281. Expression of At5g15725 was up-regulated by all three strains; however, the highest transcript levels were found after infection by the TZS-producing strain A208 (FIG. 5C) which may be related to trans-zeatin production.

To assess the effect of genes down-regulated by MTF, roots of independent T-DNA insertion mutants in At5g49520 (wrky48), At3g56710 (sigA), At4g25470 (dreb1c), At5g39670 (cbp1) and At2g43290 (mss3) were assayed. The wrky48 mutant exhibited a mild hat phenotype (FIG. 5D). None of the other tested mutants displayed increased transformation susceptibility. WRKY48 is a transcriptional activator that represses plant basal defenses (Xing et al., 2008). Results indicate that defense genes regulated by WRKY48 do not play a major role in protecting the host from Agrobacterium infection, or that Agrobacterium somehow targets and/or recruits host defenses to its advantage.

### Example 8: Effect of the MTF Mutation on Infection by Other Phytopathogens

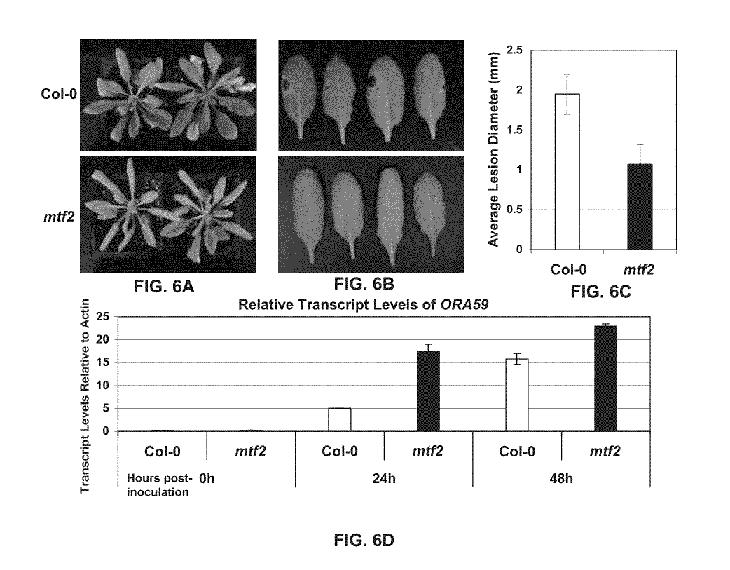

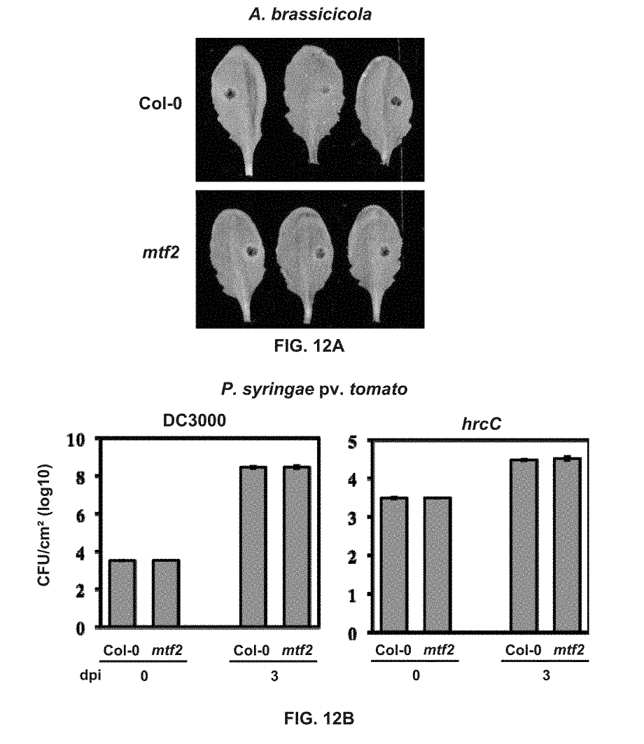

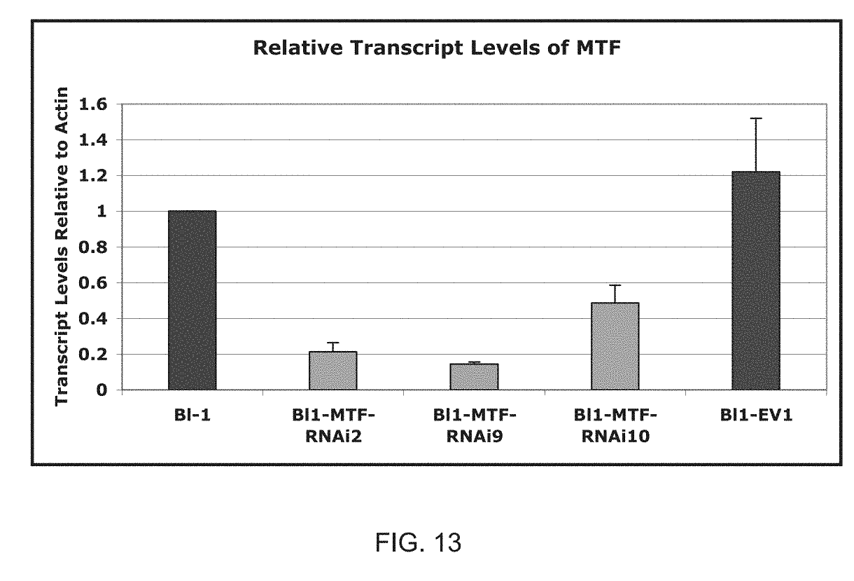

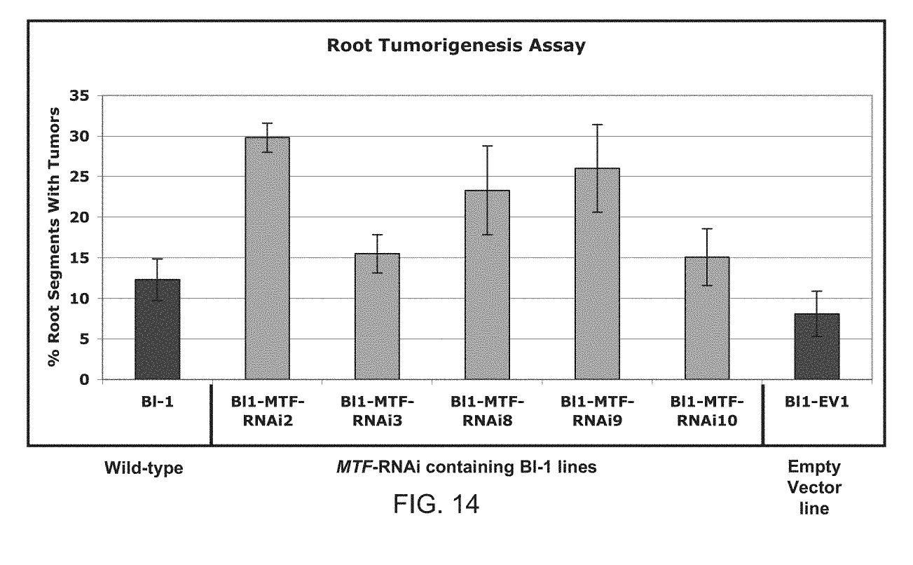

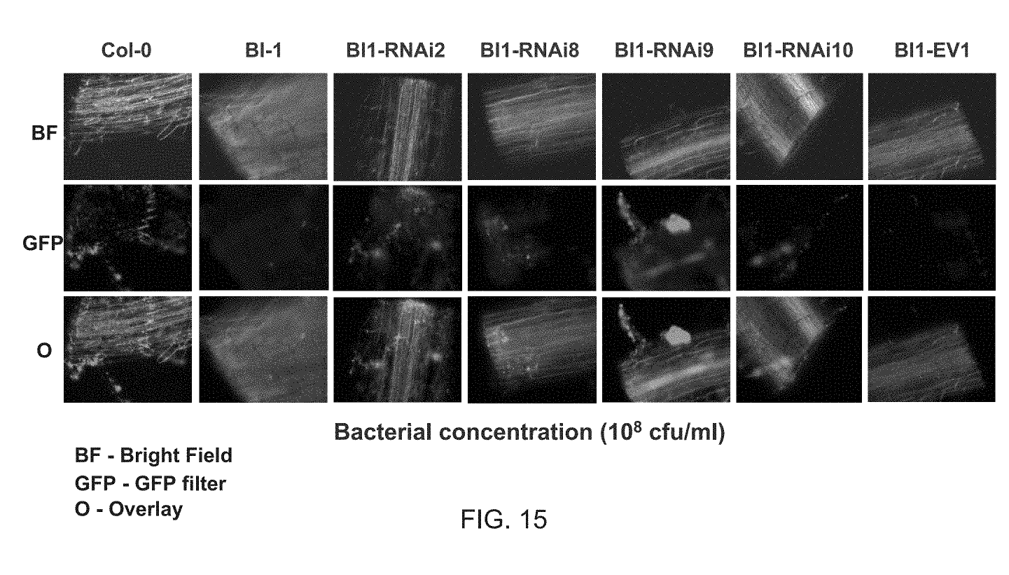

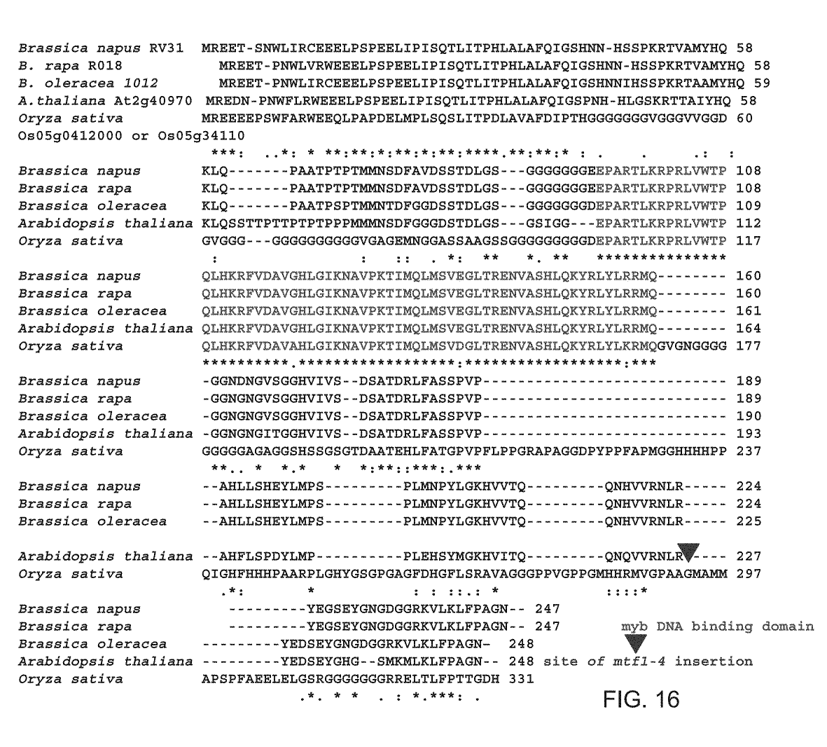

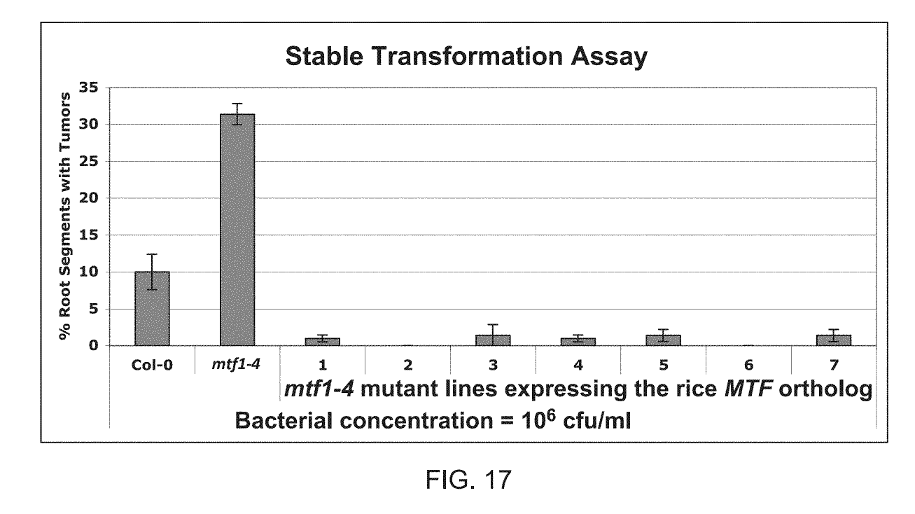

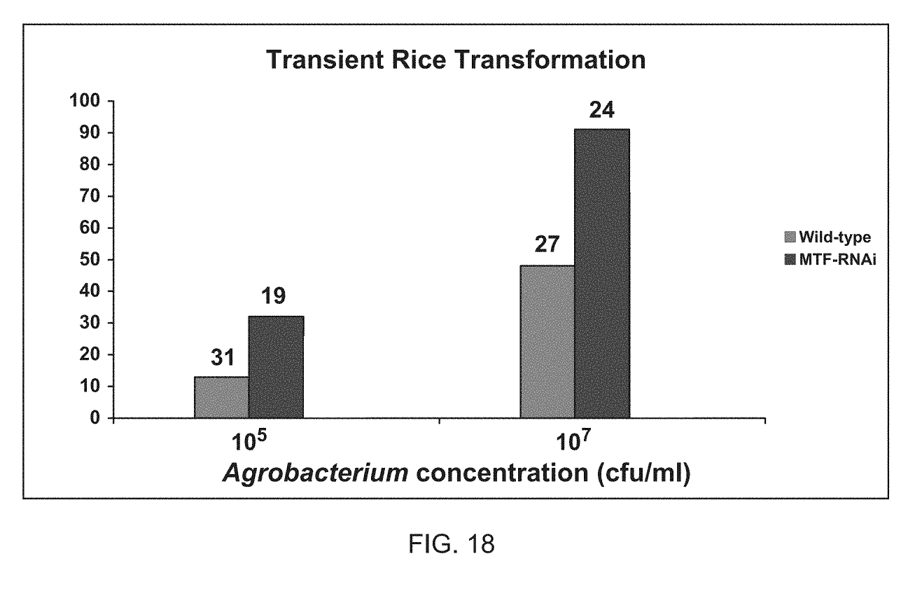

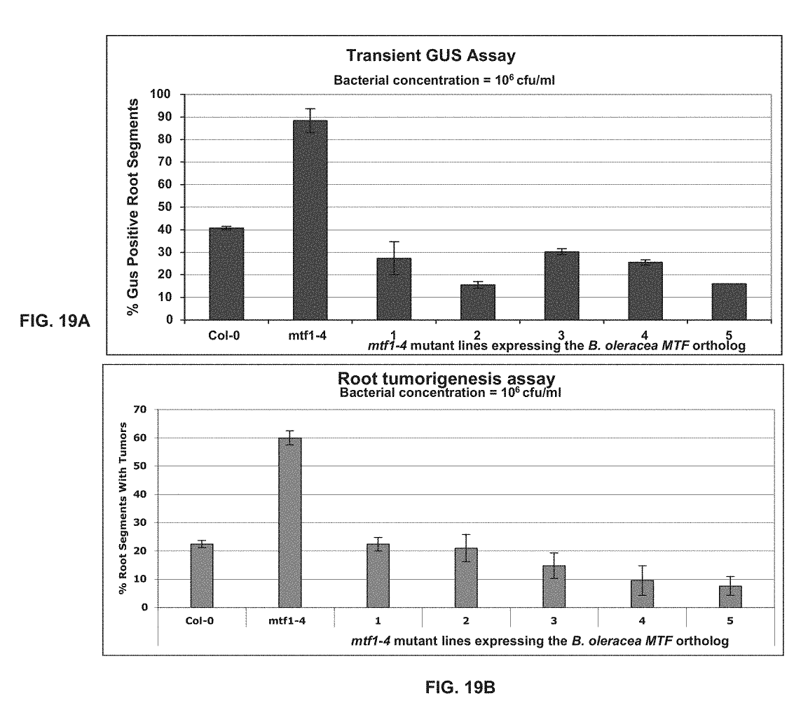

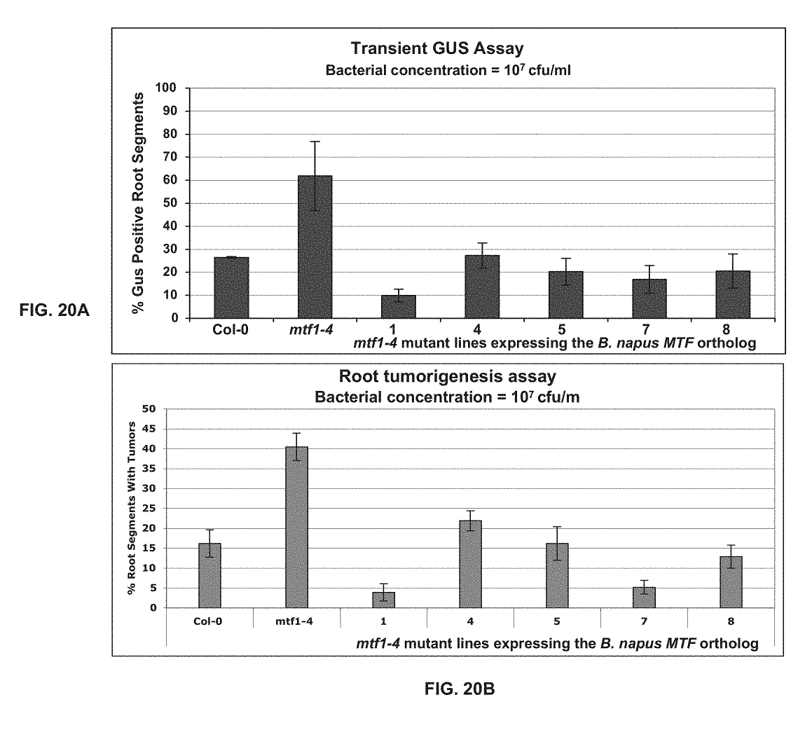

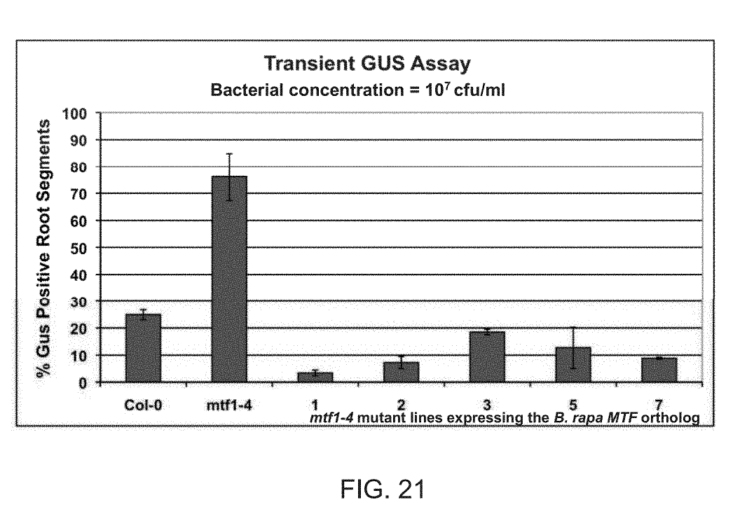

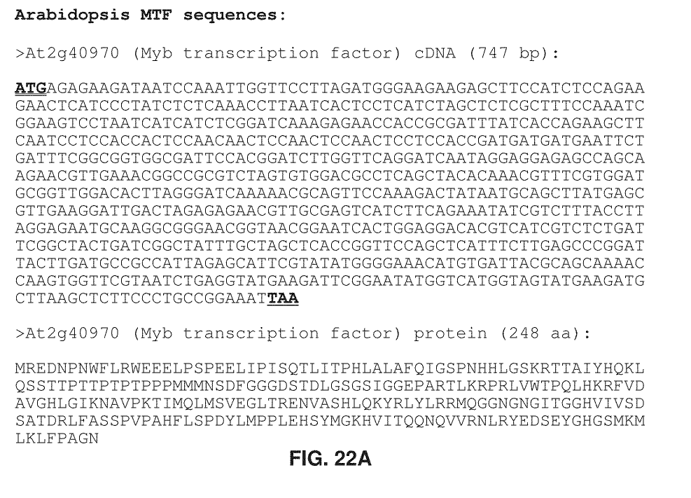

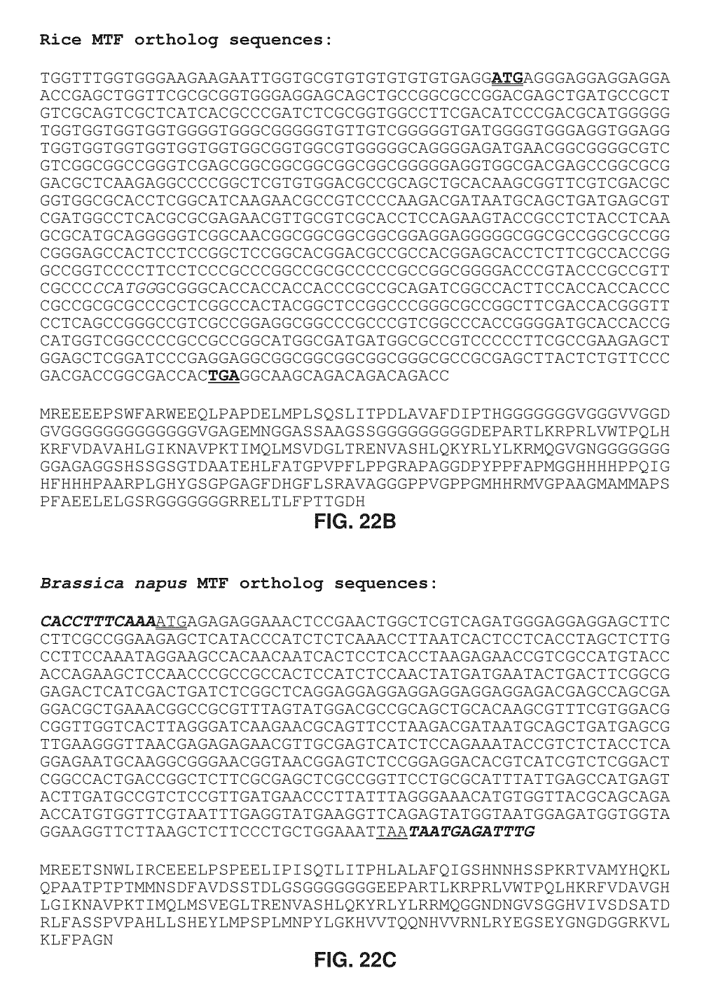

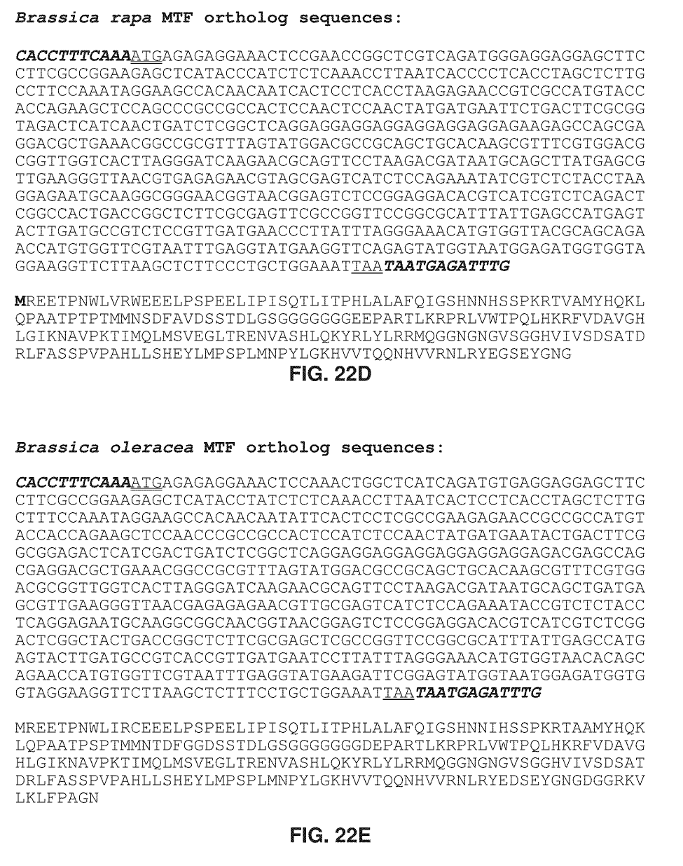

A question was whether mtf1-4 (previously mtf2) plants showed altered susceptibility to other pathogens. Col-0 and mtf1-4 (previously mtf2) plants showed similar symptoms when infected with the necrotrophic fungus Alternaria brassicicola, and the virulent DC3000 or the non-pathogenic hrcC mutant strain of Pseudomonas syringae pv. tomato (FIG. 12). However, mtf1-4 (previously mtf2) plants showed increased resistance to infection by Botrytis cinerea (FIG. 6A). Leaves of mtf1-4 (previously mtf2) drop-inoculated with B. cinerea displayed smaller lesions than did wild-type plants (FIG. 6B, C). Resistance to necrotrophic pathogens is mediated through jasmonic acid (JA) and ethylene. Microarray data revealed that At1g06160 (ORA59), encoding an octadecanoid-responsive Arabidopsis AP2/ERF transcription factor, is significantly up-regulated (1.6-fold; p<0.0001) in the mtf1-4 (previously mtf2) mutant. Because B. cinerea infection down-regulates MTF³⁵, ORA59 transcript levels were quantified in leaves of mtf1-4 (previously mtf2) and wild-type plants 0, 24, and 48 hours post-inoculation (hpi) with B. cinerea spores. By 24 hpi, more than a 3-fold increase in ORA59 transcript levels was seen in mtf1-4 (previously mtf2) compared to infected wild-type plants (FIG. 6d). Constitutive over-expression of ERF1 induces the expression of the defense-response genes PDF1.2 and ChiB (PR-3), and confers resistance to B. cinerea (Berrocal-Lobo et al., 2002). Thus, the modestly higher levels of ChiB (1.3-fold; p=0.004), and B. cinerea-induced up-regulation of ORA59 in mtf1-4 (previously mtf2), likely contribute to increased resistance to B. cinerea.

### Example 9: Manipulation of Myb Transcription Factors to Improve Crop Transformation

An Arabidopsis myb transcription factor (MTF) was identified which is a negative regulator of plant susceptibility to Agrobacterium-mediated transformation. Decreased expression of MTF results in a 10- to 15-fold increase in transformation frequency of the Arabidopsis ecotype Columbia (Col). Increased transformation susceptibility correlates with an increase in binding of Agrobacteria to the plant surface. This binding is mediated by an integrin-like protein. MTF expression is negatively regulated by cytokinins secreted by Agrobacterium cells, mediated by miaA and/or tzs.

mtf RNAi plants were generated in the transformation-recalcitrant ecotype BI-1 and transformation susceptibility was determined.

MTF orthologs were identified from crop species.

Using a bioinformatic approach i.e. “masking” the central myb DNA binding domain of MTF, and searching for proteins homologous to the N- and C-terminal regions of MTF, the correct myb orthology was verified by introducing the cDNA of an ortholog into the Arabidopsis mtf1-4 (previously mtf2) mutant and assaying for decreased transformation susceptibility.

MTF ortholog expression is identified in crop species using RNAi (or TILLING (Targeting Induced Local Lesion in Genomes)) and testing transformation susceptibility.

Results showed the following:

1. Decreased expression of MTF in A. thaliana ecotype Bl-1 results in increased Agrobacterium attachment and transformation susceptibility.

2. MTF orthologs were identified from rice and three Brassica species. The identity of these orthologs was confirmed by functional complementation of the Arabidopsis mtf1-4 (previously mtf2) mutant.

3. Decreased expression of the rice MTF ortholog by RNAi results in increased rice transformation susceptibility.

Expression of the Brassica MTF orthologs are determined in their native species and the resulting plants are assayed for increased transformation susceptibility.

Expression of the rice MTF ortholog is decreased in transformation-recalcitrant japonica and indicia lines and the resulting plants are assayed for increased transformation susceptibility.

A transient RNAi system, delivered by Agrobacterium, silences crop MTF orthologs while simultaneously delivering genes of interest to these species.

MTF orthologs from soybean and wheat were identified and are silenced. Putative orthologs were identified using bioinformatics. (using BLAST® (Basic Local Alignment Search Tool))

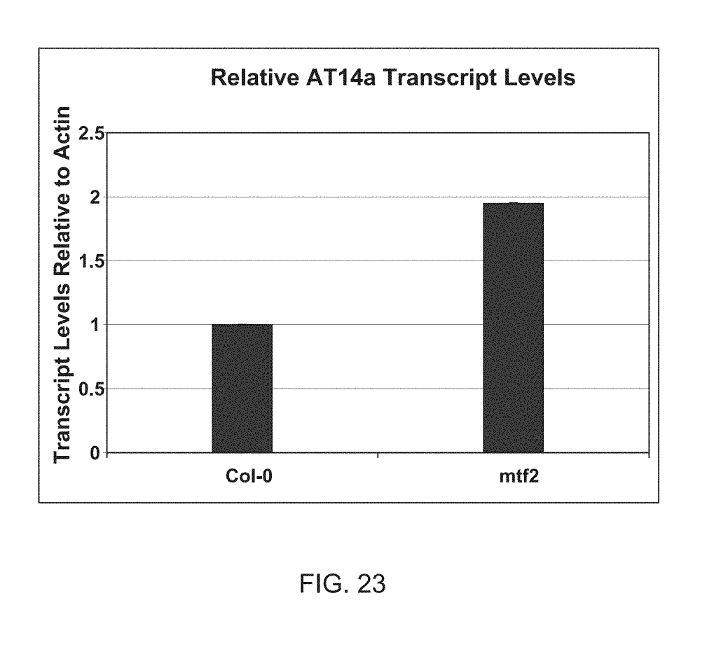

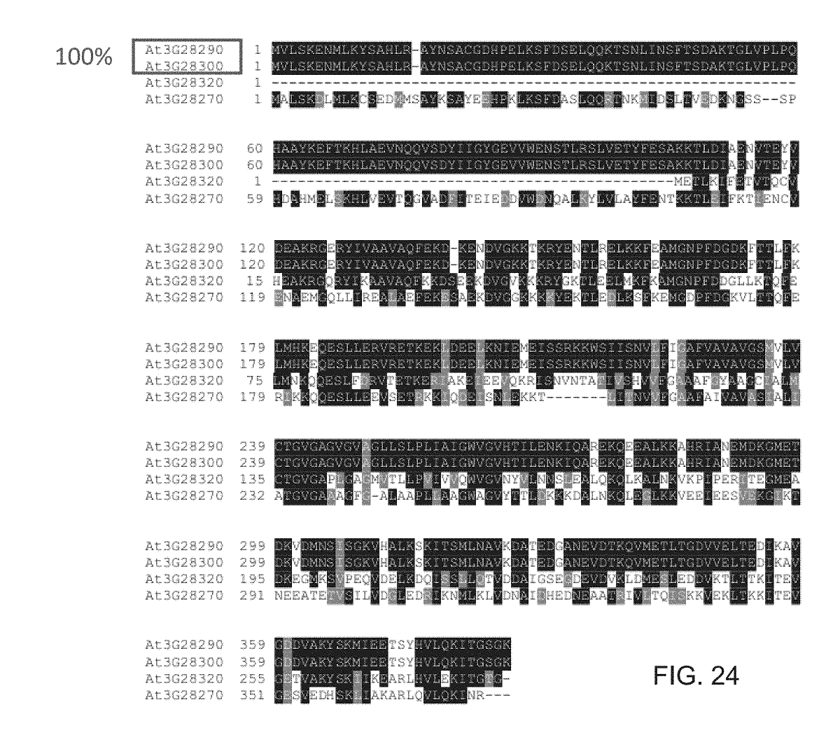

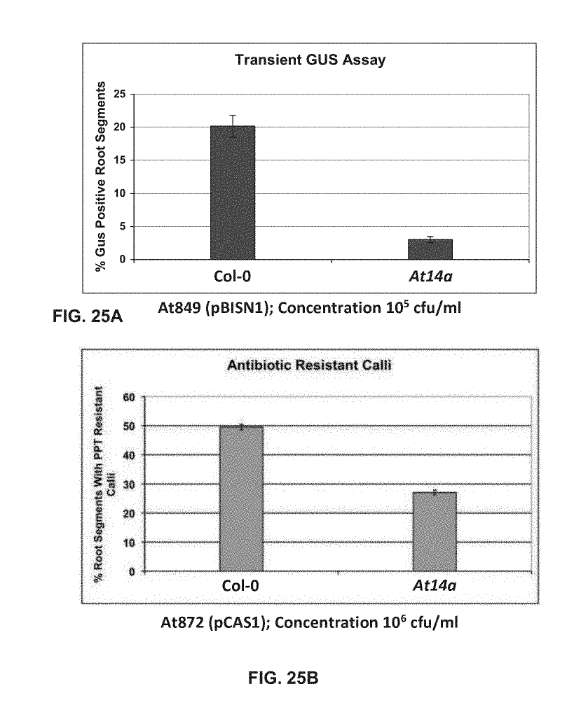

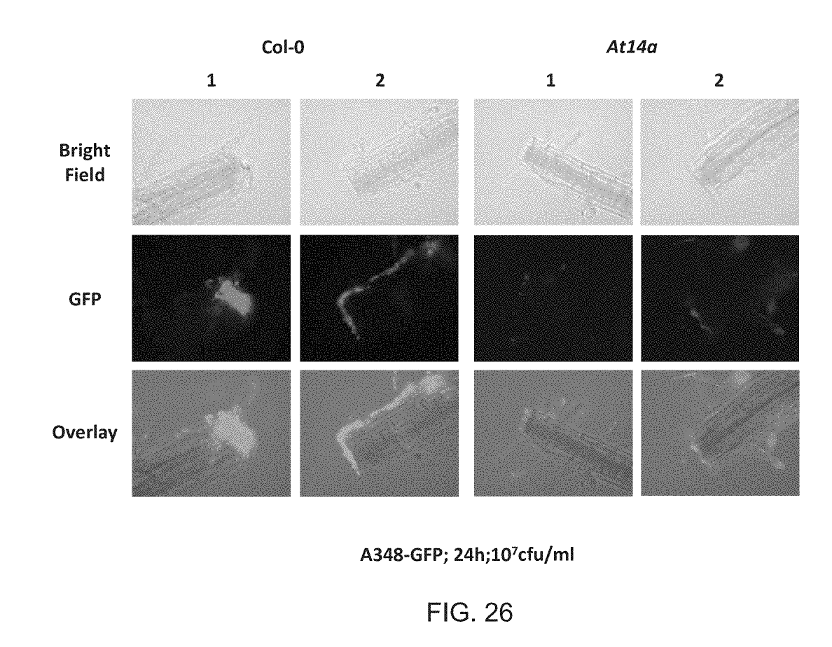

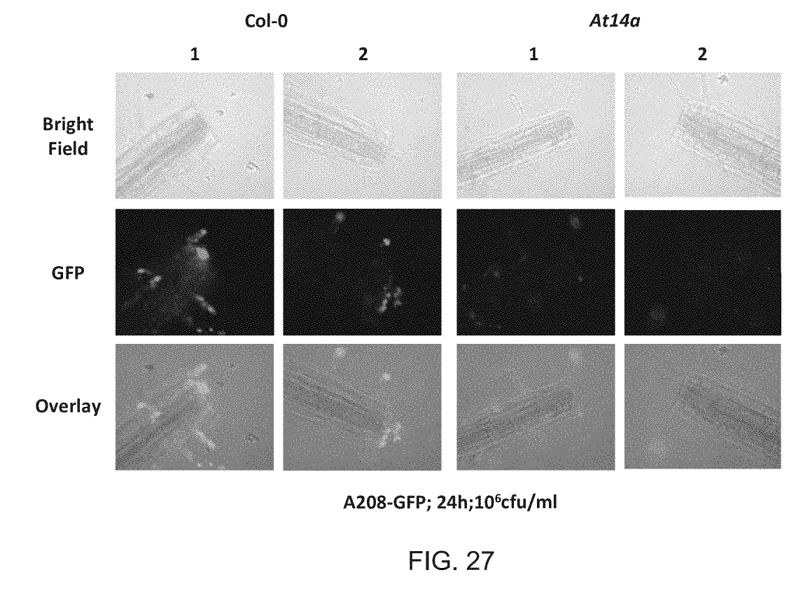

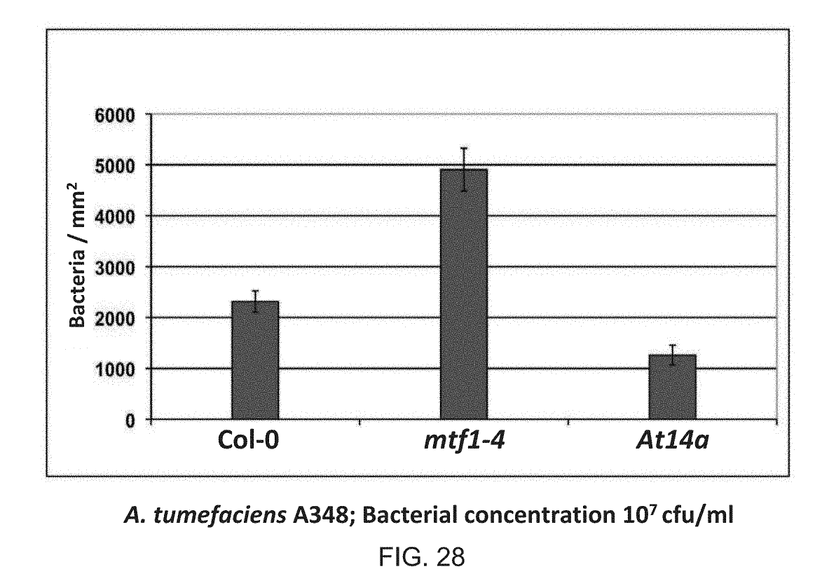

### Example 10: Involvement of the Integrin Domain-Like Protein At14a in Agrobacterium-Mediated Transformation. (See FIGS. 23, 24, 25A, 25B, 26, 27, 28)

Over-expression of the At14a gene in the Arabidopsis ecotype BI-1 increased bacterial binding to roots, and also increases root transformation. This ecotype is highly recalcitrant to Agrobacterium-mediated transformation, and binds bacteria poorly to roots.

Increasing Agrobacterium-mediated transformation of recalcitrant species, and tissues of these species, is achieved by over-expressing of the At14a gene. In particular, some tissues that are easy to regenerate but difficult to transform may not bind Agrobacterium well, and over-expressing At14a may improve binding and transformation.

### Materials and Methods

A. tumefaciens was cultured in Yeast Extract-Peptone medium (Lichtenstein et al., 1986) containing the appropriate antibiotics. Root transformation assays were carried out as previously described by Nam et al. with minor modifications (Tenea et al., 2009). MS basal medium lacking phytohormones was used to select for tumors. GUS activity assays were carried out after infection of root segments with A. tumefaciens At849 (Narasimhulu et al., 1996) for 4-6 d, using X-gluc (Jefferson et al., 1987). Detailed procedures for identifying and screening Arabidopsis mutants, generating transgenic plants, quantitative real-time RT-PCR, bacterial attachment assays, phytohormone treatment of plant roots, microarray experiments, and infection of plants with pathogenic microbes are available in the Methods.

### Agrobacterium Culture, Plant Growth Conditions and Transformation Assays

A. tumefaciens was cultured in Yeast Extract-Peptone medium containing appropriate antibiotics. Root transformation assays were carried out as previously described with minor modifications. MS basal medium lacking phytohormones was used to select for tumors. GUS activity assays were carried out after infection of root segments with A. tumefaciens At849 for 4-6 d, using X-gluc.

### Arabidopsis Mutants

˜4000 mutagenized plants from an activation-tagged library were screened at low Agrobacterium inoculation densities (10⁵ and 10⁶ cfu/mL) for increased root transformation. TAIL-PCR was utilized to identify the T-DNA/plant junction from hat3. Primers for TAIL-PCR are listed in Table 2.

Seeds of the T-DNA insertion MTF mutants SALK_072082 (mtf1), SALK_072083 (mtf1-4) (previously mtf2), and SALK_102624 (mtf3) (Alonso et al., 2003) were obtained from the Arabidopsis Biological Resource Center (Columbus, Ohio). The mutants were genotyped using primers listed in Table 2.

### Generation of Transgenic MTF-Complemented Plants

MTF cDNA was synthesized from 1-2 μg RNA using oligo(dT) and the SuperscriptIII First Strand Synthesis System for RT-PCR™ (Invitrogen, Carlsbad, Calif.), following the manufacturer's protocol. Primer sequences are listed in Table 2. The polymerase chain reaction (PCR) was conducted using PfuTurbo DNA polymerase (Stratagene, La Jolla, Calif.) and 200 ng of Arabidopsis Columbia root cDNA. PCR products were cloned into the SmaI site of pBluescript II SK+ (Stratagene). MTF cDNA was excised using XhoI and SpeI and cloned into the binary vector pE1775 (Lee et al., 2007). The resulting construction, pE3263, was introduced into A. tumefaciens GV3101 by electroporation and used for floral dip transformation (Clough and Bent, 1998) of the mutant mtf1-4. Transgenic plants were selected on B5 medium containing 20 μg/mL hygromycin.

### Quantitative Real-Time RT-PCR Analysis

Real-time RT-PCR was carried out using total RNA isolated in triplicate from roots of plants grown in liquid B5 medium. PCR was performed in triplicate on an ABI Prism 7500 Sequence Detection System (Applied Biosystems, Foster City, Calif.). Expression levels were calculated by the relative standard curve method (Applied Biosystems) for all transcripts except ORA59, where the comparative cycle threshold method (Applied Biosystems) was used, and normalized to Actin2 transcript levels. Transcript levels of genes identified in microarray experiments were validated by RT-PCR. The list of primers is given in Table 2.

### Generation of MTF-RNAi Lines

MTF-RNAi lines were generated using pFGC1008 (GenBank Accession AY310333). The RNAi construct (pE3387) contained a ˜400 bp cDNA fragment of MTF amplified using primers listed in Table 2. The MTF fragment was oriented as an inverted repeat with each repeat separated by a fragment from the gusA gene. RNAi lines, in ecotype Bl-1, were produced by floral-dip transformation using A. tumefaciens GV3101. Transgenic plants were selected on B5 medium containing hygromycin.

### Bacterial Attachment Assays

Root segments of Arabidopsis Bl-1 and MTF-RNAi lines were incubated with A. tumefaciens A208 containing pJZ383 (P_{tac}::GFP). Root segments were co-cultivated with 10⁵ cfu/mL (ecotype Columbia) or 10⁸ cfu/mL (ecotype Bl-1) for 24 h in B5 minimal medium. Root segments were rinsed and visualized by epifluorescence microscopy.

### Generation of MTF Promoter-EYFP Transgenic Plants

˜1.2 kb of the MTF promoter was amplified using a forward primer incorporating an AgeI restriction site at the 5′ end and a reverse primer incorporating the sequence for the first ten amino acids of MTF and a BamHI restriction site. Primers are listed in Table 2. The amplification product was cloned into the SmaI site of pBluescriptII SK+. The MTF promoter was excised using AgeI and BamHI and cloned into these sites of pSAT6-EYFP-N1⁴⁶ as a translational fusion with EYFP. The expression cassette was cloned as a PI-PspI fragment into pPZP-RCS2 (Tzfira et al., 2005). The resulting plasmid was transformed into A. tumefaciens GV3101 and used for floral-dip transformation of Arabidopsis Col-0. Transgenic plants were selected on B5 medium supplemented with hygromycin.

### Phytohormone Treatment of Plant Roots

Plants of Arabidopsis ecotypes Ws-2, Bl-1, Bla-2, Cal-0, Dijon-G, and Petergof were grown as described by Nam et al. and roots were excised and incubated on CIM for 0, 1, or 3 days prior to cutting into segments and infection with A. tumefaciens A208 for tumorigenesis assays or strain At849 for transient GUS expression assays.

For assessing the effect of cytokinins on MTF transcript levels and transformation, root segments from Arabidopsis Col-0 or Bl-1 were incubated on MS medium supplemented with 0, 1.4 or 14 μM trans-zeatin, and co-cultivated with either A. tumefaciens A348 or A281 for 48 h. Roots were infected with bacteria at 10⁶ cfu/mL (Col-0) or 10⁸ cfu/mL (Bl-1). Following infection, root segments were either transferred to MS basal medium containing 100 μg/mL Timentin and incubated for 4-5 weeks before recording the percentage of root segments developing tumors, or used for RNA isolation.

Agrobacterium attachment assays were conducted as described herein. Col-0 and Bl-1 root segments were co-cultivated with A281 at 10⁶ or 10⁸ cfu/mL, respectively, for 24 h in the presence or absence of 1.4 μM trans-zeatin.

### Microarray Analysis

Surface-sterilized seeds of wild-type, hat3, and mtf1-4 (previously mtf2) were germinated in B5 medium and seedlings grown for 2-weeks at 23° C. under a 16 h light/8 h dark photoperiod. Three biological replicates, each consisting of twenty seedlings of each line transferred to liquid B5 medium, were grown for 12 days. Roots were frozen in liquid N₂. RNA was isolated using Trizol reagent (Invitrogen). Microarray experiments were performed according to the Affymetrix GeneChip Expression Analysis Manual (http://www.affymetrix.com) using Arabidopsis ATH1 Genome Arrays (Affymetrix) at the Purdue University Genomics Center. GeneChip operating software was used to produce CEL files containing raw probe intensities for the arrays. Data from these files were read with “Biobase” and “affy” packages in R/Bioconductor (Gentleman et al., 2004) for analysis of genomic data. A background correction was performed on the perfect match intensities to make signals from different chips comparable. A robust local regression was employed to normalize background corrected data. An analysis of variance (ANOVA) method was employed as previously described by Chu et al., 2002, to detect probe sets which are differentially expressed between two lines using the natural log of the background corrected, normalized data as the gene expression level. To determine whether there was a statistically significant difference between two lines, it was sufficient to test whether the line effect was different from zero. This ANOVA model was performed for Col vs hat3, Col vs mtf1-4 (previously mtf2), and mtf1-4 vs hat3. Both the false discovery rate (FDR) approach (Benjamini et al., 1995) and Holm's sequential Bonferroni correction procedure (Holm, 1979) were used to adjust for multiple testing, with a significance level a of 0.05.

### Generation of Transgenic Arabidopsis Lines Over-Expressing Genes Up-Regulated in mtf Mutants

cDNAs of At2g40960, At1g50060, At5g46295, and At5g15725 were amplified using primers containing KpnI and SacI sites, and cloned into the SmaI site of pBluescriptII SK+. The primers used for amplification are listed in Table 2. DNA was digested with KpnI and SacI and cloned into pE1775 (Lee et al., 2007). The resulting constructs were introduced into A. tumefaciens GV3101 by electroporation and used for floral-dip transformation of Arabidopsis Col-0. Transgenic plants were selected on B5 medium supplemented with hygromycin.

Disease Assays on Col-0 and mtf1-4 (Previously mtf2)

Fungal and bacterial cultures were maintained and disease assays performed as previously described by Mengiste et al., 2003. Botrytis cinerea strain BO5-10 spores were harvested 10 days after initiating culture and re-suspended in 1% Sabouraud Maltose Broth (SMB) media (DIFCO, Sparks, Md.) at a concentration of 2.5×10⁵ spores/mL for spray- and drop-inoculation of whole plants. Alternaria brassicicola spores were harvested and re-suspended in distilled water at a concentration of 5×10⁵ spores/ml for drop-inoculation of detached leaves. Disease assays with Pseudomonas syringae pv. tomato DC3000 and hrcC were done as described.

TABLE 1
Fold-change of significantly differentially
regulated genes in two MTF mutants
compared to the wild-type, identified by microarray analyses
Up-regulated genes

| | | Fold change | |
|---|---|---|---|
| Gene | Annotation | hat3 | mtf1-4 |
| At1g71870 | MATE efflux family protein | 3.9 | 3.3 |
| At3g05730 | defensin-like (DEFL) family protein | 3.0 | 3.1 |
| At2g25510 | unknown protein | 1.6 | 2.6 |
| At3g16670 | phylloplanin precursor (T-phylloplanin) | 2.4 | 2.4 |
| At5g10040 | hypothetical protein | 2.6 | 2.1 |
| At2g02990 | ribonuclease, RNS1 | 2.2 | 2.0 |
| At2g41230 | similar to ARL (ARGOS-LIKE) | 1.4 | 2.0 |
| At2g40960 | nucleic acid binding | 1.5 | 1.9 |
| At1g50060 | putative pathogenesis-related protein | 1.3 | 1.8 |
| At5g46295 | expressed protein | 1.8 | 1.7 |
| At5g05900 | UGT 76C3 | 1.3 | 1.7 |
| At3g62760 | glutathione transferase III-like protein | 1.4 | 1.7 |
| At5g14750 | myb transcription factor | 1.5 | 1.7 |
| | werewolf (WER)/MYB66 | | |
| At5g15725 | expressed protein | 1.3 | 1.6 |
| At1g74490 | putative protein kinase | 1.6 | 1.6 |
| At4g38080 | putative hydroxyproline-rich | 1.9 | 1.6 |
| | glycoprotein family protein | | |
| At4g29690 | nucleotide pyrophosphatase-like protein | 1.9 | 1.6 |
| At2g25980 | jacalin lectin family protein | 1.4 | 1.5 |
| At1g74500 | putative DNA-binding bHLH protein | 1.4 | 1.5 |
| At1g23160 | GH3-like auxin-regulated protein | 1.7 | 1.5 |
| At5g44260 | zinc finger (CCCH-type) family protein | 1.3 | 1.5 |
| At2g40010 | 60S acidic ribosomal protein P0 | 1.4 | 1.5 |
| At3g17990 | phosphoethanolamine | 1.5 | 1.5 |
| | N-methyltransferase 1 | | |

Down-regulated genes

| | | | |
|---|---|---|---|
| At2g40970 | myb family transcription factor | 4.0 | 4.3 |
| At1g35210 | expressed protein | 1.4 | 2.3 |
| At1g77640 | ERF/AP2 transcription factor DREBA5 | 1.7 | 2.2 |
| At3g56710 | SigA binding protein | 1.3 | 1.9 |
| At5g37770 | calmodulin-related protein 2, | 1.3 | 1.8 |
| | touch-induced (TCH2) | | |
| At5g39670 | calcium-binding protein (CBP1) | 1.5 | 1.8 |
| At2g43290 | calmodulin-like protein (MSS3) | 1.3 | 1.8 |
| At4g25470 | DRE CRT-binding protein DREB1C | 1.4 | 1.7 |
| At5g49520 | WRKY48 | 1.3 | 1.7 |
| At4g11280 | ACC synthase (AtACS-6) | 1.3 | 1.6 |
| At1g51920 | expressed protein | 1.4 | 1.6 |
| At1g66160 | U-box domain-containing protein | 1.3 | 1.6 |
| At5g47960 | RAS superfamily GTP-binding | 1.3 | 1.6 |
| | protein (SMG1) | | |
| At1g49230 | RING-H2 finger protein RHA3a | 1.2 | 1.5 |
| At4g20000 | SigA binding protein family | 1.2 | 1.5 |

TABLE 2
Sequences of primers used (SEQ ID NOS 2-70, respectively, in
order of appearance)

| | | |
|---|---|---|
| Gene | Primer Name | Sequence (5′→3′) |

RT-PCR primers:

| | | |
|---|---|---|
| At3g18780 | Actin-FP | CTAAGCTCTCAAGATCAAAGGCTTA |
| | Actin-RP | ACTAAAACGCAAAACGAAAGCGGTT |
| | Actin2-F | GAAGTACAGTGTCTGGATCGGTGGTT |
| | Actin2-R | ATTCCTGGACCTGCCTCATCATACTC |
| At1g71870 | At1g71870-F | TGTGGTTTGGGTTGCTTTCAGCTC |
| | At1g71870-R | TCAGTCTCATTGCCTTCACGGCTT |
| At3g05730 | At3g05730-F | ATGGCAAAGACCCTCAATTCCATCTG |
| | At3g05730-R | TATTTCAACGACCGTAGCAGTGGC |
| At3g16670 | At3g16670-F | TCCTCAACATAGTCGCTATCCTCCCA |
| | At3g16670-R | GAGAAGGGAAACACACTGTAACCGAAC |
| At5g10040 | At5g10040-F | TTGCTGTGGCGGTTTCTAGTGGCTTT |
| | At5g10040-R | ACATGCCCTCTGGTGATTAGAGAAGC |
| At2g02990 | At2g02990-F | CTGGTTCCGGTTTAATCGAATGTCCG |
| | At2g02990-R | GATCGATGCCGGTTCAAGAGACTGAA |
| At2g40960 | At2g40960-F | AGCTGGTACCATGGACACAGCATTGACC |
| | At2g40960-R | CCGGGAGCTCTTACCGGTTCTGCATG |
| At2g41230 | At2g41230-F | CCTCCTCCTTCCTCTACTCCTCATGATT |
| | At2g41230-R | TTATGTATGTACGGACGGTTCGCAACGC |
| At5g46295 | At5g46295-F | TGAGAAGATGATGAGAAAAGGGAAGCTTTC |
| | At5g46295-R | TGTTAGAATTTACAACCACAACAGAGGAAG |
| At1g50060 | At1g50060-F | CAGTGAAGATAGGGTGTGCTAGGGTT |
| | At1g50060-R | ATCAGTAAGGGTACTCTCCGACCCAA |
| At3g62760 | At3g62760-F | ATCTCCACCACGTGCCTTACACTTAC |
| | At3g62760-R | TTAAGGAAAGCCGGACGAGAACAGAG |
| At5g14750 | At5g14750-F | TGGGTTCATGAGGATGAGTTTGAGC |
| | At5g14750-R | GACTGTTGATGTATTAGTGTTTGATCAGC |
| At5g15725 | At5g15725-F | CGACCAAGGATATAATATGAAGAAGACGAG |
| | At5g15725-R | GTCAATTAGTGACGATTACGCACGCC |
| At1g74490 | At1g74490-F | TTTAGTCCTTAGGATGTCTGAGAAACCC |
| | At1g74490-R | GGTTAGACCATCGATGCTTGAGGT |
| At4g38080 | At4g38080-F | GCCCACAATCCCTAACATTCCACAGA |
| | At4g38080-R | AGTGTGTGATCCAAAGCTGTCTCAGG |
| At1g35210 | At1g35210-F | GGTTTGGTAATGGGCACAAAGAAGAG |
| | At1g35210-R | CTTGCACGTACCCACCAAACTGATCT |
| At1g77640 | At1g77640-F | CGGAGATCCGTTTGATTATTCTCCAC |
| | At1g77640-R | TGGACCGTTGGATTAACTGAAACTCC |
| At3g56710 | At3g56710-F | GTGATTGTTATGAGCCGTTGAATGCGG |
| | At3g56710-R | TCACATAGAATCGATGCTTCCAAAGTCA |
| At5g37770 | At5g37770-F | GTGAGAAGTGCTCTGTGCAAGATTGT |
| | At5g37770-R | CGGCGAAATCTTCCAAATCCTCAAGC |
| At5g39670 | At5g39670-F | CGATGGAAGTAAAGACGGAAGAATCG |
| | At5g39670-R | GGTGCGGAGACAACAGTATTAACAGAC |
| At2g43290 | At2g43290-F | AGGTGGTGGCTTTAGCAGCAGTA |
| | At2g43290-R | ACACCTTCCTCGATTACACGATGTT |
| At4g25470 | At4g25470-F | TTGATGTCGAGGGAGATGATGACGTG |
| | At4g25479-R | ACCATTTACATTCGTTTCTCACAACCAA |
| At5g49520 | At5g49520-F | CCTTCGCAGATCAGATCCGATACTATT |
| | At5g49520-R | ACTCCTCATGAAACCTACCTACCGGA |
| At4g11280 | At4g11280-F | GAAGAAGTGTTGGCAGAGTAACCTCAG |
| | At4g11280-R | TCTGTGCACGGACTAGCGGAGAA |

### TAIL-PCR Primers:

### Degenerate Primers:

AD1:
NTCASTWTWTSGWGTT
AD2:
NGTCGASWGANAWGAA
AD3:
WGTGNAGWANCANAGA

pSKI015-Specific Primers:

ACT-TAIL1:
TGGATTGATGTGATATCTAGATCCG
ACT-TAIL2:
CCCCCACCCACGAGGAACATCGTGG
ACT-TAIL3:
GGAAGATGGCTTCTACAAATGCCAT

### Primers to Genotype MTF Mutant Plants:

MTF-RT forward:
CTCATCCCTATCTCTCAAACC
MTF reverse:
TTCCGGCAGGGAAGAGCTTAAGCATCTT
T-DNA primer LBa1:
TGGTTCACGTAGTGGGCCATCG

Primers to Amplify MTF cDNA:

MTF-XhoI-F
ACGGCTCGAGATGAGAGAAGATAATCCA
MTF-SpeI-R
AACCACTAGTTTAATTTCCGGCAGGGAAG

### Real-Time RT-PCR Primers:

MTF-RT forward
CTCATCCCTATCTCTCAAACC
MTF-RT reverse
TCTGAAGATGACTCGCAACGT
qORA59-F
TCGCGGCCGAGATAAGAGACTC
qORA59-R
TCCGGAGAGATTCTTCAACGACATCC

### MTF RNAi Primers:

MTF-RNAi-F
ACACTAGTGGCGCGCCTTTACCTTAGGAGAATGC
MTF-RNAi-R
ACGGATCCATTTAAATTTGATCCTGACGACAAAT

### MTF Promoter Primers:

MybPro-AgeI:
CCCCACCGGTATACTACAAAATACCTAAAACAAAATGT
MybPro-BamHI:
CCAAGGATCCGAGATGGAAGCTCTTCTTC

### PUBLICATIONS CITED

These publications are incorporated by reference to the extent they relate materials and methods disclosed herein.
Aly, K. A., et al. J. Bacteriol. 190, 1595-1604 (2008).
Beaty, J. S. et al. Mol. Gen. Genet 203, 274-280 (1986).
Benjamini, Y. & Hochberg, Y. J. Roy. Statist. Soc. Ser. B (Methodol.) 57, 289-300 (1995).
Berrocal-Lobo, M., et al. Plant J. 29, 23-32 (2002).
Chateau, S., et al. J. Exp. Bot. 51, 1961-1968 (2000).
Chu, T.-M., et al. Mathematical Biosci. 17, 35-51 (2002).
Claeys, M., et al. Fresenius Z. Anal. Chem. 290, 125-126 (1978).
Clough, S. J. & Bent, A. F. Plant J. 16, 735-743 (1998).
Fernandez, C. et al. J. Mol. Biol. 266, 576-593 (1997).
Gentleman, R. C., et al. Genome Biol. 5, R80 (2004).
Hazen, S. P. et al. Proc. Natl. Acad. Sci. 102, 10387-10392 (2005).
Holm, S. Scand. J. Stat. 6, 65-70 (1979).
Hwang, H.-H. et al. Mol. Plant Pathol. 11, 677-690 (2010).
Jefferson, R. A., et al. EMBO J. 6, 3901-3907 (1987).
Lacroix, B., et al. Trends Genet. 22, 29-37 (2006).
Lee, L.-Y. et al. Plant Physiol. 145, 1294-1300 (2007).
Lichtenstein, C. & Draper, J. Genetic engineering of plants. In DNA Cloning. A Practical Approach (ed. D. M. Glover), Vol. 2, 67-119 (Washington, D.C., IRL Press, 1986).
Liu, Y.-G., et al. Plant J. 8, 457-463 (1995).
McCloskey, J. A., et al. FEBS Lett. 111, 181-183 (1980).
McCullen, C. A. & Binns, A. N. Annu. Rev. Cell Dev. Biol. 22, 101-127 (2006).
Mengiste, T., et al. Plant Cell 15, 2551-2565 (2003).
Milne, T. J., et al. J. Biol. Chem. 278, 31105-31110 (2003).
Mysore, K. S., et al. Plant J. 21, 9-16 (2000).
Nam, J. et al. Mol. Gen. Genet. 261, 429-438 (1999).
Nam, J., et al. Plant Cell 9, 317-333 (1997).
Narasimhulu, S. B., et al. Plant Cell 8, 873-886 (1996).
Niki, T., et al. Plant Cell Physiol 39, 500-507 (1998).
Powell, G. K., et al. Mol Plant-Microbe Interact. 1, 235-242 (1988).
Regier, D. A. & Morris, R. O. Biochem. Biophys. Res. Commun. 104, 1560-1566 (1982).
Tenea, G. N. et al. Plant Cell 21, 3350-3367 (2009).
Tzfira, T. et al. Plant Mol. Biol. 57, 503-516 (2005).
Valvekens, D., et al. Proc. Natl. Acad. Sci. USA 85: 5536-5540 (1988).
Veena, Jiang, H., et al. Plant J. 35, 219-236 (2003).
Villemont, E., et al. Planta 201, 160-172 (1997).
Weigel, D. et al. Plant Physiol. 122, 1003-1013 (2000).
Xing, D.-H. et al. Mol. Plant 1, 459-470 (2008).
Zhan, X., et al. Plant Mol. Biol. 14, 785-792 (1990).
Zhu, Y. et al. Plant Physiol. 132, 494-505 (2003).
Zimmermann, P., Hirsch-Hoffmann, M., Hennig, L. & Gruissem, W. GENEVESTIGATOR. Arabidopsis microarray database and analysis toolbox. Plant Physiol. 136, 2621-2632 (2004).

1. Functional orthologs in plants of a family of myb transcription factors designated MTF in Arabidopsis, wherein the factors negatively regulate plant transformation susceptibility.

2. A myb transcription factor of claim 1 wherein the MTF is designated At2g40970.

3. The myb transcription factor of claim 1 wherein the plant is selected from the group consisting of rice, Brassica species, wheat, maize, and soybean.

4. The myb transcription factor of claim 3, wherein the plant is rice.

5. A mutant plant with increased susceptibility to Agrobacterium-mediated transformation, the mutant having the hat3 phenotype and a functional ortholog of claim 1.

6. A method to increase transformation in a plant, the method comprises use of ortholog of claim 1.

7. The method of claim 6 wherein the transformation is stable.


SEQUENCE LISTING (93 sequences)

SEQ ID NO: 1 - protein, 7 aa, Arabidopsis sp.
SHAQKYF

SEQ ID NO: 2 - DNA, 25 bp, Artificial Sequence
  Description of Artificial Sequence Synthetic primer
ctaagctctcaagatcaaaggctta

SEQ ID NO: 3 - DNA, 25 bp, Artificial Sequence
  Description of Artificial Sequence Synthetic primer
actaaaacgcaaaacgaaagcggtt

SEQ ID NO: 4 - DNA, 26 bp, Artificial Sequence
  Description of Artificial Sequence Synthetic primer
gaagtacagtgtctggatcggtggtt

SEQ ID NO: 5 - DNA, 26 bp, Artificial Sequence
  Description of Artificial Sequence Synthetic primer
attcctggacctgcctcatcatactc

SEQ ID NO: 6 - DNA, 24 bp, Artificial Sequence
  Description of Artificial Sequence Synthetic primer
tgtggtttgggttgctttcagctc

SEQ ID NO: 7 - DNA, 24 bp, Artificial Sequence
  Description of Artificial Sequence Synthetic primer
tcagtctcattgccttcacggctt

SEQ ID NO: 8 - DNA, 26 bp, Artificial Sequence
  Description of Artificial Sequence Synthetic primer
atggcaaagaccctcaattccatctg

SEQ ID NO: 9 - DNA, 24 bp, Artificial Sequence
  Description of Artificial Sequence Synthetic primer
tatttcaacgaccgtagcagtggc

SEQ ID NO: 10 - DNA, 26 bp, Artificial Sequence
  Description of Artificial Sequence Synthetic primer
tcctcaacatagtcgctatcctccca

SEQ ID NO: 11 - DNA, 27 bp, Artificial Sequence
  Description of Artificial Sequence Synthetic primer
gagaagggaaacacactgtaaccgaac

SEQ ID NO: 12 - DNA, 26 bp, Artificial Sequence
  Description of Artificial Sequence Synthetic primer
ttgctgtggcggtttctagtggcttt

SEQ ID NO: 13 - DNA, 26 bp, Artificial Sequence
  Description of Artificial Sequence Synthetic primer
acatgccctctggtgattagagaagc

SEQ ID NO: 14 - DNA, 26 bp, Artificial Sequence
  Description of Artificial Sequence Synthetic primer
ctggttccggtttaatcgaatgtccg

SEQ ID NO: 15 - DNA, 26 bp, Artificial Sequence
  Description of Artificial Sequence Synthetic primer
gatcgatgccggttcaagagactgaa

SEQ ID NO: 16 - DNA, 28 bp, Artificial Sequence
  Description of Artificial Sequence Synthetic primer
agctggtaccatggacacagcattgacc

SEQ ID NO: 17 - DNA, 26 bp, Artificial Sequence
  Description of Artificial Sequence Synthetic primer
ccgggagctcttaccggttctgcatg

SEQ ID NO: 18 - DNA, 28 bp, Artificial Sequence
  Description of Artificial Sequence Synthetic primer
cctcctccttcctctactcctcatgatt

SEQ ID NO: 19 - DNA, 28 bp, Artificial Sequence
  Description of Artificial Sequence Synthetic primer
ttatgtatgtacggacggttcgcaacgc

SEQ ID NO: 20 - DNA, 30 bp, Artificial Sequence
  Description of Artificial Sequence Synthetic primer
tgagaagatgatgagaaaagggaagctttc

SEQ ID NO: 21 - DNA, 30 bp, Artificial Sequence
  Description of Artificial Sequence Synthetic primer
tgttagaatttacaaccacaacagaggaag

SEQ ID NO: 22 - DNA, 26 bp, Artificial Sequence
  Description of Artificial Sequence Synthetic primer
cagtgaagatagggtgtgctagggtt

SEQ ID NO: 23 - DNA, 26 bp, Artificial Sequence
  Description of Artificial Sequence Synthetic primer
atcagtaagggtactctccgacccaa

SEQ ID NO: 24 - DNA, 26 bp, Artificial Sequence
  Description of Artificial Sequence Synthetic primer
atctccaccacgtgccttacacttac

SEQ ID NO: 25 - DNA, 26 bp, Artificial Sequence
  Description of Artificial Sequence Synthetic primer
ttaaggaaagccggacgagaacagag

SEQ ID NO: 26 - DNA, 25 bp, Artificial Sequence
  Description of Artificial Sequence Synthetic primer
tgggttcatgaggatgagtttgagc

SEQ ID NO: 27 - DNA, 29 bp, Artificial Sequence
  Description of Artificial Sequence Synthetic primer
gactgttgatgtattagtgtttgatcagc

SEQ ID NO: 28 - DNA, 30 bp, Artificial Sequence
  Description of Artificial Sequence Synthetic primer
cgaccaaggatataatatgaagaagacgag

SEQ ID NO: 29 - DNA, 26 bp, Artificial Sequence
  Description of Artificial Sequence Synthetic primer
gtcaattagtgacgattacgcacgcc

SEQ ID NO: 30 - DNA, 28 bp, Artificial Sequence
  Description of Artificial Sequence Synthetic primer
tttagtccttaggatgtctgagaaaccc

SEQ ID NO: 31 - DNA, 24 bp, Artificial Sequence
  Description of Artificial Sequence Synthetic primer
ggttagaccatcgatgcttgaggt

SEQ ID NO: 32 - DNA, 26 bp, Artificial Sequence
  Description of Artificial Sequence Synthetic primer
gcccacaatccctaacattccacaga

SEQ ID NO: 33 - DNA, 26 bp, Artificial Sequence
  Description of Artificial Sequence Synthetic primer
agtgtgtgatccaaagctgtctcagg

SEQ ID NO: 34 - DNA, 26 bp, Artificial Sequence
  Description of Artificial Sequence Synthetic primer
ggtttggtaatgggcacaaagaagag

SEQ ID NO: 35 - DNA, 26 bp, Artificial Sequence
  Description of Artificial Sequence Synthetic primer
cttgcacgtacccaccaaactgatct

SEQ ID NO: 36 - DNA, 26 bp, Artificial Sequence
  Description of Artificial Sequence Synthetic primer
cggagatccgtttgattattctccac

SEQ ID NO: 37 - DNA, 26 bp, Artificial Sequence
  Description of Artificial Sequence Synthetic primer
tggaccgttggattaactgaaactcc

SEQ ID NO: 38 - DNA, 27 bp, Artificial Sequence
  Description of Artificial Sequence Synthetic primer
gtgattgttatgagccgttgaatgcgg

SEQ ID NO: 39 - DNA, 28 bp, Artificial Sequence
  Description of Artificial Sequence Synthetic primer
tcacatagaatcgatgcttccaaagtca

SEQ ID NO: 40 - DNA, 26 bp, Artificial Sequence
  Description of Artificial Sequence Synthetic primer
gtgagaagtgctctgtgcaagattgt

SEQ ID NO: 41 - DNA, 26 bp, Artificial Sequence
  Description of Artificial Sequence Synthetic primer
cggcgaaatcttccaaatcctcaagc

SEQ ID NO: 42 - DNA, 26 bp, Artificial Sequence
  Description of Artificial Sequence Synthetic primer
cgatggaagtaaagacggaagaatcg

SEQ ID NO: 43 - DNA, 27 bp, Artificial Sequence
  Description of Artificial Sequence Synthetic primer
ggtgcggagacaacagtattaacagac

SEQ ID NO: 44 - DNA, 23 bp, Artificial Sequence
  Description of Artificial Sequence Synthetic primer
aggtggtggctttagcagcagta

SEQ ID NO: 45 - DNA, 25 bp, Artificial Sequence
  Description of Artificial Sequence Synthetic primer
acaccttcctcgattacacgatgtt

SEQ ID NO: 46 - DNA, 26 bp, Artificial Sequence
  Description of Artificial Sequence Synthetic primer
ttgatgtcgagggagatgatgacgtg

SEQ ID NO: 47 - DNA, 28 bp, Artificial Sequence
  Description of Artificial Sequence Synthetic primer
accatttacattcgtttctcacaaccaa

SEQ ID NO: 48 - DNA, 27 bp, Artificial Sequence
  Description of Artificial Sequence Synthetic primer
ccttcgcagatcagatccgatactatt

SEQ ID NO: 49 - DNA, 26 bp, Artificial Sequence
  Description of Artificial Sequence Synthetic primer
actcctcatgaaacctacctaccgga

SEQ ID NO: 50 - DNA, 27 bp, Artificial Sequence
  Description of Artificial Sequence Synthetic primer
gaagaagtgttggcagagtaacctcag

SEQ ID NO: 51 - DNA, 23 bp, Artificial Sequence
  Description of Artificial Sequence Synthetic primer
tctgtgcacggactagcggagaa

SEQ ID NO: 52 - DNA, 16 bp, Artificial Sequence
  Description of Artificial Sequence Synthetic primer
  modified_base (1)..(1): a, c, t, g, unknown or other
ntcastwtwtsgwgtt

SEQ ID NO: 53 - DNA, 16 bp, Artificial Sequence
  Description of Artificial Sequence Synthetic primer
  modified_base (1)..(1): a, c, t, g, unknown or other
  modified_base (11)..(11): a, c, t, g, unknown or other
ngtcgaswganawgaa

SEQ ID NO: 54 - DNA, 16 bp, Artificial Sequence
  Description of Artificial Sequence Synthetic primer
  modified_base (5)..(5): a, c, t, g, unknown or other
  modified_base (10)..(10): a, c, t, g, unknown or other
  modified_base (13)..(13): a, c, t, g, unknown or other
wgtgnagwancanaga

SEQ ID NO: 55 - DNA, 25 bp, Artificial Sequence
  Description of Artificial Sequence Synthetic primer
tggattgatgtgatatctagatccg

SEQ ID NO: 56 - DNA, 25 bp, Artificial Sequence
  Description of Artificial Sequence Synthetic primer
cccccacccacgaggaacatcgtgg

SEQ ID NO: 57 - DNA, 25 bp, Artificial Sequence
  Description of Artificial Sequence Synthetic primer
ggaagatggcttctacaaatgccat

SEQ ID NO: 58 - DNA, 21 bp, Artificial Sequence
  Description of Artificial Sequence Synthetic primer
ctcatccctatctctcaaacc

SEQ ID NO: 59 - DNA, 28 bp, Artificial Sequence
  Description of Artificial Sequence Synthetic primer
ttccggcagggaagagcttaagcatctt

SEQ ID NO: 60 - DNA, 22 bp, Artificial Sequence
  Description of Artificial Sequence Synthetic primer
tggttcacgtagtgggccatcg

SEQ ID NO: 61 - DNA, 28 bp, Artificial Sequence
  Description of Artificial Sequence Synthetic primer
acggctcgagatgagagaagataatcca

SEQ ID NO: 62 - DNA, 29 bp, Artificial Sequence
  Description of Artificial Sequence Synthetic primer
aaccactagtttaatttccggcagggaag

SEQ ID NO: 63 - DNA, 21 bp, Artificial Sequence
  Description of Artificial Sequence Synthetic primer
ctcatccctatctctcaaacc

SEQ ID NO: 64 - DNA, 21 bp, Artificial Sequence
  Description of Artificial Sequence Synthetic primer
tctgaagatgactcgcaacgt

SEQ ID NO: 65 - DNA, 22 bp, Artificial Sequence
  Description of Artificial Sequence Synthetic primer
tcgcggccgagataagagactc

SEQ ID NO: 66 - DNA, 26 bp, Artificial Sequence
  Description of Artificial Sequence Synthetic primer
tccggagagattcttcaacgacatcc

SEQ ID NO: 67 - DNA, 34 bp, Artificial Sequence
  Description of Artificial Sequence Synthetic primer
acactagtggcgcgcctttaccttaggagaatgc

SEQ ID NO: 68 - DNA, 34 bp, Artificial Sequence
  Description of Artificial Sequence Synthetic primer
acggatccatttaaatttgatcctgacgacaaat

SEQ ID NO: 69 - DNA, 38 bp, Artificial Sequence
  Description of Artificial Sequence Synthetic primer
ccccaccggtatactacaaaatacctaaaacaaaatgt

SEQ ID NO: 70 - DNA, 29 bp, Artificial Sequence
  Description of Artificial Sequence Synthetic primer
ccaaggatccgagatggaagctcttcttc

SEQ ID NO: 71 - protein, 247 aa, Brassica napus
MREETSNWLIRCEEELPSPEELIPISQTLITPHLALAFQIGSHNNHSSPKRTVAMYHQKLQPAATPTPTMMNSDFAVDSSTDLGSGGGGGGGEEPARTLKRPRLVWTPQLHKRFVDAVGHLGIKNAVPKTIMQLMSVEGLTRENVASHLQKYRLYLRRMQGGNDNGVSGGHVIVSDSATDRLFASSPVPAHLLSHEYLMPSPLMNPYLGKHVVTQQNHVVRNLRYEGSEYGNGDGGRKVLKLFPAGN

SEQ ID NO: 72 - protein, 247 aa, Brassica rapa
MREETPNWLVRWEEELPSPEELIPISQTLITPHLALAFQIGSHNNHSSPKRTVAMYHQKLQPAATPTPTMMNSDFAVDSSTDLGSGGGGGGGEEPARTLKRPRLVWTPQLHKRFVDAVGHLGIKNAVPKTIMQLMSVEGLTRENVASHLQKYRLYLRRMQGGNGNGVSGGHVIVSDSATDRLFASSPVPAHLLSHEYLMPSPLMNPYLGKHVVTQQNHVVRNLRYEGSEYGNGDGGRKVLKLFPAGN

SEQ ID NO: 73 - protein, 248 aa, Brassica oleracea
MREETPNWLIRCEEELPSPEELIPISQTLITPHLALAFQIGSHNNIHSSPKRTAAMYHQKLQPAATPSPTMMNTDFGGDSSTDLGSGGGGGGGDEPARTLKRPRLVWTPQLHKRFVDAVGHLGIKNAVPKTIMQLMSVEGLTRENVASHLQKYRLYLRRMQGGNGNGVSGGHVIVSDSATDRLFASSPVPAHLLSHEYLMPSPLMNPYLGKHVVTQQNHVVRNLRYEDSEYGNGDGGRKVLKLFPAGN

SEQ ID NO: 74 - protein, 248 aa, Arabidopsis thaliana
MREDNPNWFLRWEEELPSPEELIPISQTLITPHLALAFQIGSPNHHLGSKRTTAIYHQKLQSSTTPTTPTPTPPPMMMNSDFGGGDSTDLGSGSIGGEPARTLKRPRLVWTPQLHKRFVDAVGHLGIKNAVPKTIMQLMSVEGLTRENVASHLQKYRLYLRRMQGGNGNGITGGHVIVSDSATDRLFASSPVPAHFLSPDYLMPPLEHSYMGKHVITQQNQVVRNLRYEDSEYGHGSMKMLKLFPAGN

SEQ ID NO: 75 - protein, 331 aa, Oryza sativa
MREEEEPSWFARWEEQLPAPDELMPLSQSLITPDLAVAFDIPTHGGGGGGGVGGGVVGGDGVGGGGGGGGGGGGGVGAGEMNGGASSAAGSSGGGGGGGGGDEPARTLKRPRLVWTPQLHKRFVDAVAHLGIKNAVPKTIMQLMSVDGLTRENVASHLQKYRLYLKRMQGVGNGGGGGGGGGAGAGGSHSSGSGTDAATEHLFATGPVPFLPPGRAPAGGDPYPPFAPMGGHHHHPPQIGHFHHHPAARPLGHYGSGPGAGFDHGFLSRAVAGGGPPVGPPGMHHRMVGPAAGMAMMAPSPFAEELELGSRGGGGGGGRRELTLFPTTGDH

SEQ ID NO: 76 - DNA, 747 bp, Arabidopsis thaliana
atgagagaagataatccaaattggttccttagatgggaagaagagcttccatctccagaagaactcatccctatctctcaaaccttaatcactcctcatctagctctcgctttccaaatcggaagtcctaatcatcatctcggatcaaagagaaccaccgcgatttatcaccagaagcttcaatcctccaccactccaacaactccaactccaactcctccaccgatgatgatgaattctgatttcggcggtggcgattccacggatcttggttcaggatcaataggaggagagccagcaagaacgttgaaacggccgcgtctagtgtggacgcctcagctacacaaacgtttcgtggatgcggttggacacttagggatcaaaaacgcagttccaaagactataatgcagcttatgagcgttgaaggattgactagagagaacgttgcgagtcatcttcagaaatatcgtctttaccttaggagaatgcaaggcgggaacggtaacggaatcactggaggacacgtcatcgtctctgattcggctactgatcggctatttgctagctcaccggttccagctcatttcttgagcccggattacttgatgccgccattagagcattcgtatatggggaaacatgtgattacgcagcaaaaccaagtggttcgtaatctgaggtatgaagattcggaatatggtcatggtagtatgaagatgcttaagctcttccctgccggaaattaa

SEQ ID NO: 77 - DNA, 1059 bp, Oryza sativa
tggtttggtgggaagaagaattggtgcgtgtgtgtgtgtgaggatgagggaggaggaggaaccgagctggttcgcgcggtgggaggagcagctgccggcgccggacgagctgatgccgctgtcgcagtcgctcatcacgcccgatctcgcggtggccttcgacatcccgacgcatgggggtggtggtggtggtggggtgggcgggggtgttgtcgggggtgatggggtgggaggtggaggtggtggtggtggtggtggtggcggtggcgtgggggcaggggagatgaacggcggggcgtcgtcggcggccgggtcgagcggcggcggcggcggcgggggaggtggcgacgagccggcgcggacgctcaagaggccccggctcgtgtggacgccgcagctgcacaagcggttcgtcgacgcggtggcgcacctcggcatcaagaacgccgtccccaagacgataatgcagctgatgagcgtcgatggcctcacgcgcgagaacgttgcgtcgcacctccagaagtaccgcctctacctcaagcgcatgcagggggtcggcaacggcggcggcggcggaggagggggcggcgccggcgccggcgggagccactcctccggctccggcacggacgccgccacggagcacctcttcgccaccgggccggtccccttcctcccgcccggccgcgcccccgccggcggggacccgtacccgccgttcgcccccatgggcgggcaccaccaccacccgccgcagatcggccacttccaccaccaccccgccgcgcgcccgctcggccactacggctccggcccgggcgccggcttcgaccacgggttcctcagccgggccgtcgccggaggcggcccgcccgtcggcccaccggggatgcaccaccgcatggtcggccccgccgccggcatggcgatgatggcgccgtcccccttcgccgaagagctggagctcggatcccgaggaggcggcggcggcggcgggcgccgcgagcttactctgttcccgacgaccggcgaccactgaggcaagcagacagacagacc

SEQ ID NO: 78 - DNA, 767 bp, Brassica napus
cacctttcaaaatgagagaggaaactccgaactggctcgtcagatgggaggaggagcttccttcgccggaagagctcatacccatctctcaaaccttaatcactcctcacctagctcttgccttccaaataggaagccacaacaatcactcctcacctaagagaaccgtcgccatgtaccaccagaagctccaacccgccgccactccatctccaactatgatgaatactgacttcggcggagactcatcgactgatctcggctcaggaggaggaggaggaggaggagacgagccagcgaggacgctgaaacggccgcgtttagtatggacgccgcagctgcacaagcgtttcgtggacgcggttggtcacttagggatcaagaacgcagttcctaagacgataatgcagctgatgagcgttgaagggttaacgagagagaacgttgcgagtcatctccagaaataccgtctctacctcaggagaatgcaaggcgggaacggtaacggagtctccggaggacacgtcatcgtctcggactcggccactgaccggctcttcgcgagctcgccggttcctgcgcatttattgagccatgagtacttgatgccgtctccgttgatgaacccttatttagggaaacatgtggttacgcagcagaaccatgtggttcgtaatttgaggtatgaaggttcagagtatggtaatggagatggtggtaggaaggttcttaagctcttccctgctggaaattaataatgagatttg

SEQ ID NO: 79 - DNA, 767 bp, Brassica rapa
cacctttcaaaatgagagaggaaactccgaaccggctcgtcagatgggaggaggagcttccttcgccggaagagctcatacccatctctcaaaccttaatcacccctcacctagctcttgccttccaaataggaagccacaacaatcactcctcacctaagagaaccgtcgccatgtaccaccagaagctccagcccgccgccactccaactccaactatgatgaattctgacttcgcggtagactcatcaactgatctcggctcaggaggaggaggaggaggaggagaagagccagcgaggacgctgaaacggccgcgtttagtatggacgccgcagctgcacaagcgtttcgtggacgcggttggtcacttagggatcaagaacgcagttcctaagacgataatgcagcttatgagcgttgaagggttaacgtgagagaacgtagcgagtcatctccagaaatatcgtctctacctaaggagaatgcaaggcgggaacggtaacggagtctccggaggacacgtcatcgtctcagactcggccactgaccggctcttcgcgagttcgccggttccggcgcatttattgagccatgagtacttgatgccgtctccgttgatgaacccttatttagggaaacatgtggttacgcagcagaaccatgtggttcgtaatttgaggtatgaaggttcagagtatggtaatggagatggtggtaggaaggttcttaagctcttccctgctggaaattaataatgagatttg

SEQ ID NO: 80 - DNA, 770 bp, Brassica oleracea
cacctttcaaaatgagagaggaaactccaaactggctcatcagatgtgaggaggagcttccttcgccggaagagctcatacctatctctcaaaccttaatcactcctcacctagctcttgctttccaaataggaagccacaacaatattcactcctcgccgaagagaaccgccgccatgtaccaccagaagctccaacccgccgccactccatctccaactatgatgaatactgacttcggcggagactcatcgactgatctcggctcaggaggaggaggaggaggaggagacgagccagcgaggacgctgaaacggccgcgtttagtatggacgccgcagctgcacaagcgtttcgtggacgcggttggtcacttagggatcaagaacgcagttcctaagacgataatgcagctgatgagcgttgaagggttaacgagagagaacgttgcgagtcatctccagaaataccgtctctacctcaggagaatgcaaggcggcaacggtaacggagtctccggaggacacgtcatcgtctcggactcggctactgaccggctcttcgcgagctcgccggttccggcgcatttattgagccatgagtacttgatgccgtcaccgttgatgaatccttatttagggaaacatgtggtaacacagcagaaccatgtggttcgtaatttgaggtatgaagattcggagtatggtaatggagatggtggtaggaaggttcttaagctctttcctgctggaaattaataatgagatttg

SEQ ID NO: 81 - protein, 385 aa, Arabidopsis sp.
MVLSKENMLKYSAHLRAYNSACGDHPELKSFDSELQQKTSNLINSFTSDAKTGLVPLPQHAAYKEFTKHLAEVNQQVSDYIIGYGEVVWENSTLRSLVETYFESAKKTLDIAENVTEYVDEAKRGERYIVAAVAQFEKDKENDVGKKTKRYENTLRELKKFEAMGNPFDGDKFTTLFKLMHKEQESLLERVRETKEKLDEELKNIEMEISSRKKWSIISNVLFIGAFVAVAVGSMVLVCTGVGAGVGVAGLLSLPLIAIGWVGVHTILENKIQAREKQEEALKKAHRIANEMDKGMETDKVDMNSISGKVHALKSKITSMLNAVKDATEDGANEVDTKQVMETLTGDVVELTEDIKAVGDDVAKYSKMIEETSYHVLQKITGSGK

SEQ ID NO: 82 - protein, 280 aa, Arabidopsis sp.
METLKIFETVTQCVHEAKRGQRYIKAAVAQFKKDSEEKDVGVKKKRYGKTLEELMKFKAMGNPFDDGLLKTQFELMNKQQESLFDRVTETKERIAKEIEEVQKRISNVNTATIVSHVVFGAAAFGYAAGCIALMCTGVGAPLGAGMVTLLPVIVVQWVGVNYVLNNSLEALQKQLKALNKVKPIPERITEGMEADKEGMKSVPEQVDELKDQISSLLQTVDDAIGSEGDEVDVKLDMESLEDDVKTLTTKITEVGETVAKYSKIIKEARLHVLEKITGTG

SEQ ID NO: 83 - protein, 374 aa, Arabidopsis sp.
MALSKDLMLKCSEDMMSACKSACEEHPKLKSFDASLQQRTNKMIDSLTVEDKNGSSSPHDAHMELSKHLVEVTQGVADFITEIEDDVWDNQALKYLVLAYFENTKKTLEIFKTIENCVENAEMGQLLIREALAEFEKESAEKDVGGKKKKYEKTLEDLKSFKEMGDPFDGKVLTTQFERIKKQQESLLEEVSETRKKIQDEISNLEKKTLITNVVFGAAFAIVAVASIALIATGVGAAAGFGALAAPLLAAGWAGVYTTLDKKKDALNKQLEGLKKVEEIEESVEKGIKTNEEATETVSILVDGLEDRIKNMLKLVDNAIDHEDNEAATRIVLTQISKKVEKLTKKITEVGESVEDHSKLIAKARLQVLQKINR

SEQ ID NO: 84 - protein, 9 aa, Oryza sativa
HHHPPQIGH

SEQ ID NO: 85 - protein, 4 aa, Oryza sativa
IGHF

SEQ ID NO: 86 - protein, 5 aa, Oryza sativa
AAGMA

SEQ ID NO: 87 - protein, 8 aa, Oryza sativa
PPQIGHFH

SEQ ID NO: 88 - DNA, 396 bp, Oryza sativa
cacctcttcgccaccgggccggtccccttcctcccgcccggccgcgcccccgccggcggggacccgtacccgccgttcgcccccatgggcgggcaccaccaccacccgccgcagatcggccacttccaccaccaccccgccgcgcgcccgctcggccactacggctccggcccgggcgccggcttcgaccacgggttcctcagccgggccgtcgccggaggcggcccgcccgtcggcccaccggggatgcaccaccgcatggtcggccccgccgccggcatggcgatgatggcgccgtcccccttcgccgaagagctggagctcggatcccgaggaggcggcggcggcggcgggcgccgcgagcttactctgttcccgacgaccggcgaccactga

SEQ ID NO: 89 - protein, 131 aa, Oryza sativa
HLFATGPVPFLPPGRAPAGGDPYPPFAPMGGHHHHPPQIGHFHHHPAARPLGHYGSGPGAGFDHGFLSRAVAGGGPPVGPPGMHHRMVGPAAGMAMMAPSPFAEELELGSRGGGGGGGRRELTLFPTTGDH

SEQ ID NO: 90 - protein, 95 aa, Oryza sativa
TSSPPGRSPSSRPAAPPPAGTRTRRSPPWAGTTTTRRRSATSTTTPPRARSATTAPARAPASTTGSSAGPSPEAARPSAHRGCTTAWSAPPPAWR

SEQ ID NO: 91 - protein, 35 aa, Oryza sativa
WRRPPSPKSWSSDPEEAAAAAGAASLLCSRRPATT

SEQ ID NO: 92 - protein, 131 aa, Oryza sativa
PLRHRAGPLPPARPRPRRRGPVPAVRPHGRAPPPPAADRPLPPPPRRAPARPLRLRPGRRLRPRVPQPGRRRRRPARRPTGDAPPHGRPRRRHGDDGAVPLRRRAGARIPRRRRRRRAPRAYSVPDDRRPL

SEQ ID NO: 93 - protein, 6 aa, Artificial Sequence
  Description of Artificial Sequence Synthetic 6xHis tag
HHHHHH